(19) United States
(12) Patent Application Publication
(10) Pub. No.: US 2025/0384952 A1
(43) Pub. Date: Dec. 18, 2025

(54) TANDEM REPEAT GENOTYPING

(71) Applicant: Illumina, Inc., San Diego, CA (US)

(72) Inventors: Qi Wang, San Diego, CA (US); Suzanne Rohrback, San Diego, CA (US); Mitchell A. Bekritsky, Decatur, GA (US); Asli Yildirim, San Diego, CA (US)

(21) Appl. No.: 18/878,513

(22) PCT Filed: Mar. 29, 2024
(86) PCT No.: PCT/US2024/022265

§ 371 Date: Dec. 23, 2024

Related U.S. Application Data
(60) Provisional application No. 63/493,081, filed on Mar. 30, 2023.

(51) Int. Cl. G16B 20/20

(52) U.S. Cl. CPC G16B 20/20

(57) ABSTRACT

This disclosure describes methods, non-transitory-computer readable media, and systems that can accurately generate genotypes for tandem-repeat regions of a genomic sample by utilizing an expectation-maximization (EM) algorithm and a stutter model. The disclosed system can extract spanning nucleotide reads that comprise whole tandem-repeat regions. The disclosed system may perform an expectation stage of an EM algorithm and utilize a stutter model to predict expected genotype probabilities of tandem-repeat genotypes given a distribution of spanning reads. In some implementations, the disclosed system further performs a maximization stage of the EM algorithm to adjust parameters of the stutter model based on the expected genotype probabilities to maximize a total probability of the expected genotype probabilities. The disclosed system can repeat the expectation and maximization stages until the total probability of the expected genotype probabilities converges. The disclosed system may predict a genotype for the tandem repeat based on the converged genotype probabilities.

### CROSS-REFERENCE TO RELATED APPLICATIONS

The present application claims the benefit of, and priority to, U.S. Provisional Application No. 63/493,081, titled, “SHORT TANDEM REPEAT (STR) GENOTYPING,” filed Mar. 30, 2023. The aforementioned application is hereby incorporated by reference in its entirety.

### BACKGROUND

In recent years, biotechnology firms and research institutions have improved hardware and software for sequencing nucleotides and determining nucleobase calls for genomic samples. For instance, some existing sequencing machines and sequencing-data-analysis software (together “existing sequencing systems”) predict individual nucleobases within sequences by using conventional Sanger sequencing or sequencing-by-synthesis (SBS) methods. When using SBS, existing sequencing systems can monitor many thousands to millions of oligonucleotides being synthesized in parallel from templates to predict nucleobase calls for growing nucleotide reads. In many existing sequencing systems, a camera captures images of irradiated fluorescent tags incorporated into oligonucleotides. After capturing such images, some existing sequencing systems determine nucleobase calls for nucleotide reads corresponding to the oligonucleotides and send base-call data to a computing device with sequencing-data-analysis software, which aligns nucleotide reads with a reference genome. Based on differences between the aligned nucleotide reads and the reference genome, existing systems can further utilize a variant caller to identify variants of a genomic sample, such as single nucleotide polymorphisms (SNPs), and/or special-purpose callers to predict genotypes in tandem-repeat regions for the genomic sample, such as Short Tandem Repeat (STR) or microsatellite regions or minisatellite regions.

Accurately identifying tandem-repeat genotypes is important for clinical treatment and improving human health in part because STR expansions, Variable Number Tandem Repeat (VNTR) expansions, and other tandem-repeat expansions cause many diseases. However, the repetitive sequences of tandem-repeat regions frequently cause alignment errors that bias downstream analyses. Furthermore, PCR stutter errors often result in reads having more or fewer repeat units than the true genotype. Some existing tandem-repeat genotyping systems have been designed to determine tandem-repeat genotypes, such as STR genotypes or microsatellite regions or minisatellite regions. These existing tandem-repeat genotyping systems primarily detect STRs or VNTRs from PCR-free whole-genome sequencing. In some examples, existing tandem-repeat genotyping systems utilize population-scale sequencing data to mine candidate tandem-repeat alleles. These existing tandem-repeat genotyping systems may utilize specialized models to align sample reads containing STRs or VNTRs to the candidate alleles while accounting for STR or VNTR artifacts. Existing tandem-repeat genotyping systems may further integrate population-scale SNP data and phased SNP haplotypes to predict likely sample tandem-repeat genotypes.

Despite these recent advances, existing sequencing systems and tandem-repeat genotyping systems face several shortcomings. For example, existing systems frequently determine inaccurate tandem-repeat genotypes from PCR-reliant data. During PCR amplification, DNA polymerase slippage events can add or delete copies of repeat units. Existing systems often fail to take into consideration errors originating from PCR amplification. Because of their inability to account for stutter artifacts, existing sequencing systems often generate inaccurate tandem-repeat genotype predictions. Because most methylation assays implement a PCR amplification step, existing systems are often incapable of accurately predicting tandem-repeat genotypes using methylation data.

In addition to inaccurate tandem-repeat genotyping, existing tandem-repeat genotyping systems often have limited application to population samples. To illustrate, because existing tandem-repeat genotyping systems rely on population-scale data, they typically generate most likely alleles for populations. While existing systems may identify common mutations within large population sizes, they are often incapable of tandem-repeat genotyping individual samples.

In addition to accuracy and sampling challenges, some existing sequencing and tandem-repeat genotyping systems inefficiently rely on an inordinate amount of input to genotype STRs or VNTRs. Existing tandem-repeat genotyping systems often rely on and devote computing resources to analyzing population data. Additionally, existing systems often require SNP or VNTR calling information, and more specifically, phased SNP or VNTR haplotype data to determine corresponding genotypes. Furthermore, some existing systems rely on in-frame and out-of-frame read classifications. The requirement of excessive amounts of data is computationally expensive and often prohibitive. Thus, existing systems often rely on a significant amount of data and computer processing resources to determine tandem-repeat genotypes for a single genomic sequence.

These, along with additional problems and issues exist in existing sequencing and tandem-repeat genotyping systems.

### SUMMARY

This disclosure describes one or more embodiments of systems, methods, and non-transitory computer readable storage media that solve one or more of the problems described above or provide other advantages over the art. The disclosed systems can improve tandem-repeat genotype calling accuracy from methylation data by utilizing a stutter model to predict tandem-repeat genotypes based on spanning reads. In some implementations, the disclosed systems extract spanning reads that cover entire tandem-repeat regions for a genomic sample, such as Short Tandem Repeat (STR) or Variable Number Tandem Repeat (VNTR) regions. Given the differing repeat units among spanning reads and a stutter model, the disclosed systems can calculate an expected probabilities of tandem-repeat genotypes for the genomic sample. Based on the expected genotype probabilities for a given iteration, the disclosed systems further update parameters of the stutter model and re-calculate the expected tandem-repeat genotype probabilities until the tandem-repeat genotype probabilities converge. The disclosed systems subsequently predict a tandem-repeat genotype for the genomic sample based on the converged tandem-repeat genotype probabilities.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description refers to the drawings briefly described below.

FIG. 1 illustrates an environment in which a tandem-repeat genotype sequencing system can operate in accordance with one or more embodiments of the present disclosure.

FIG. 2 illustrates an overview of an expectation-maximization (EM) algorithm intuition utilized by the tandem-repeat genotype sequencing system in accordance with one or more embodiments of the present disclosure.

FIG. 3 illustrates an overview the tandem-repeat genotype sequencing system determining a genotype call for a tandem-repeat region in accordance with one or more implementations of the present disclosure.

FIG. 4 illustrates the tandem-repeat genotype sequencing system generating candidate STR genotypes in accordance with one or more embodiments of the present disclosure.

FIG. 5 illustrates the tandem-repeat genotype sequencing system determining initial genotype probabilities in accordance one or more embodiments of the present disclosure.

FIG. 6 illustrates the tandem-repeat genotype sequencing system performing an expectation stage of an EM algorithm and determining expected genotype probabilities in accordance with one or more embodiments of the present disclosure.

FIGS. 7A-7B illustrate the tandem-repeat genotype sequencing system performing a maximization stage of an EM algorithm and updating the parameters of the stutter model in accordance with one or more embodiments of the present disclosure.

FIG. 8 illustrates the tandem-repeat genotype sequencing system determining the genotype call from candidate STR genotypes in accordance with one or more embodiments of the present disclosure.

FIGS. 9A-9D illustrate a series of graphs indicating the tandem-repeat genotype sequencing system more accurately genotyping STRs from methylation data relative to existing sequencing systems in accordance with one or more embodiments of the present disclosure.

FIG. 10 illustrates a flowchart of a series of acts of determining genotype calls from candidate STR genotypes in accordance with one or more embodiments of the present disclosure.

FIG. 11 illustrates a block diagram of an example computing device in accordance with one or more embodiments of the present disclosure.

### DETAILED DESCRIPTION

This disclosure describes one or more embodiments of a tandem-repeat genotype sequencing system that can accurately determine a genotype of one or more tandem-repeat regions from a genomic sample by (i) identifying spanning reads covering a tandem-repeat region from reads of the genomic sample and (ii) utilizing a stutter model to iteratively predict tandem-repeat genotype probabilities and update the stutter model's parameters based on spanning reads. In some implementations, the tandem-repeat genotype sequencing system extracts, from the reads in methylation sequencing assay for a genomic sample, spanning reads comprising entire tandem-repeat regions. Such regions may include Short Tandem Repeat (STR) or microsatellites, minisatellites, Variable Number Tandem Repeat (VNTR), guanine-quadruplexes, or other tandem-repeat regions. In an expectation stage of an expectation-maximization (EM) algorithm, the tandem-repeat genotype sequencing system can utilize a stutter model to calculate an expected probability of tandem-repeat genotypes given the spanning reads. In a maximization stage of the EM algorithm, the tandem-repeat genotype sequencing system can update parameters of the stutter model. By iteratively repeating the expectation and maximization stages, the tandem-repeat genotype sequencing system adjusts the tandem-repeat genotype probabilities until convergence. The tandem-repeat genotype sequencing system subsequently predicts a tandem-repeat genotype for the genomic sample based on the converged tandem-repeat genotype probabilities.

As just noted, the tandem-repeat genotype sequencing system can extract, from a set of nucleotide reads sequenced for a genomic sample, spanning nucleotide reads of a genomic sample. In some embodiments, for example, the tandem-repeat genotype sequencing system identifies a subset of nucleotide reads that cover a tandem-repeat region from nucleotide reads sequenced for a genomic sample. The tandem-repeat genotype sequencing system predicts tandem-repeat genotypes utilizing this limited sample of spanning nucleotide reads. As explained below, the tandem-repeat genotype sequencing system may further determine candidate tandem-repeat genotypes based on the spanning reads.

After extracting the spanning reads for a genomic sample, the tandem-repeat genotype sequencing system can initialize data for an EM algorithm. In particular, the tandem-repeat genotype sequencing system initializes allele probabilities and genotype probabilities for individual tandem repeats based on the differing numbers of repeat units in the extracted spanning reads. In addition to initializing allele and genotype probabilities, in some embodiments, the tandem-repeat genotype sequencing system initializes values for a stutter model by initializing (i) a relatively higher value for an increased-repeat-unity probability of a given nucleotide read comprising more repeat units than a reference tandem-repeat region and (ii) a relatively lower value for a decreased-repeat-unit probability of the given nucleotide read comprising fewer repeat units than the reference tandem-repeat region. Unlike previous tandem-repeat genotyping systems that ignore real-world data, such a higher increased-repeat-unity probability relative to a lower decreased-repeat-unit probability better reflects real-world proportions and leads to improved accuracy.

Having initialized probabilities and stutter-model parameters, the tandem-repeat genotype sequencing system can execute a unique EM algorithm. For instance, the tandem-repeat genotype sequencing system can perform an expectation stage of an EM algorithm to generate expected genotype probabilities of candidate tandem-repeat genotypes. In some implementations, the tandem-repeat genotype sequencing system utilizes a stutter model to generate expected genotype probabilities based on differing numbers of nucleotide repeat units in the spanning nucleotide reads.

After the expectation stage, the tandem-repeat genotype sequencing system can perform a maximization stage of the EM algorithm to update parameters of the stutter model. As indicated above, for instance, the tandem-repeat genotype sequencing system can update (u) an increased-repeat-unity probability of a given nucleotide read comprising more repeat units than a reference tandem-repeat region, (d) a decreased-repeat-unit probability of the given nucleotide read comprising fewer repeat units than the reference tandem-repeat region, and (q) a size of stutter-induced changes. In some embodiments, the tandem-repeat genotype sequencing system modifies the parameters of the stutter model to maximize a total probability of the expected genotype probabilities.

After an initial expectation stage and maximization stage, in some cases, the tandem-repeat genotype sequencing system iteratively repeats both stages of the EM algorithm until reaching converged genotype probabilities of tandem-repeat genotypes. For example, after a first iteration, the tandem-repeat genotype sequencing system utilizes the stutter model having updated parameters to generate updated allele and genotype probabilities for a tandem-repeat region.

In some implementations, the tandem-repeat genotype sequencing system determines a genotype call from the candidate tandem-repeat genotypes based on the converged genotype probabilities. For instance, the tandem-repeat genotype sequencing system can select the candidate tandem-repeat genotype having the highest total probability as a most probable tandem-repeat genotype.

As indicated above, the tandem-repeat genotype sequencing system provides several technical advantages relative to existing sequencing systems by, for example, improving genotyping accuracy, genotyping specificity, and computational efficiency relative to existing sequencing systems. For example, the tandem-repeat genotype sequencing system improves the accuracy of tandem-repeat genotyping by accounting for PCR stutter errors. More specifically, the tandem-repeat genotype sequencing system utilizes the stutter model to estimate variations in different numbers of nucleotide repeat units resulting from error, as shown in spanning nucleotide reads sequenced for a genomic sample in a methylation sequencing assay. By identifying spanning nucleotide reads covering a tandem-repeat region from methylation sequencing reads of a genomic sample—and utilizing a stutter model to iteratively predict tandem-repeat genotype probabilities and update the stutter model's parameters based on different repeat units exhibited by spanning nucleotide reads—the tandem-repeat genotype sequencing system determines more accurate genotype calls in tandem-repeat regions for genomic samples than existing methylation sequencing systems. Because most current methylation assays require PCR amplification steps, the tandem-repeat genotype sequencing system can substantially improve tandem-repeat genotype calling accuracies from methylation data. In some examples, the tandem-repeat genotype sequencing system makes a 3% improvement to genotype calling accuracy and decreases inaccurate genotype calling by 30% relative to existing methylation sequencing systems.

Beyond improved genotyping accuracy, in some embodiments, the tandem-repeat genotype sequencing system improves specificity relative to existing methylation sequencing systems. More specifically, while some existing sequencing systems are designed for population samples, the tandem-repeat genotype sequencing system can be designed to predict tandem-repeat genotypes for single samples. Due in part to its efficient utilization of spanning reads, the tandem-repeat genotype sequencing system generates accurate tandem-repeat genotypes specific to individual samples. By identifying spanning nucleotide reads covering a tandem-repeat region—and identifying differing numbers of nucleotide repeat units among the spanning nucleotide reads—the tandem-repeat genotype sequencing system can leverage the spanning nucleotide reads for a particular genomic sample (rather than a population of different genomic samples) to execute an EM algorithm for determining genotype calls for the particular genomic sample's tandem-repeat region.

In some implementations, the tandem-repeat genotype sequencing system improves efficiency in processing and data input relative to existing methylation sequencing systems. In contrast to existing methylation sequencing systems that require SNP calling information, the tandem-repeat genotype sequencing system can accurately predict SNP genotypes in tandem-repeat regions based on spanning nucleotide reads and the number of nucleotide repeat units in each spanning read as input for a genomic sample—but without SNP calls. Furthermore, while existing systems typically require additional classifications of read data, for instance, including phasing data for reads associated with a tandem-repeat region and in-frame and out-of-of frame classifications for partial or full nucleotide repeat units within such reads, the tandem-repeat genotype sequencing system simplifies the prediction process by removing in-frame and out-of-frame classifications. Rather than such in-frame and out-of-frame classifications, the tandem-repeat genotype sequencing system processes and leverages the spanning nucleotide reads of a genomic sample. In contrast to existing sequencing systems that require data from multiple assays or sources, in some embodiments, the tandem-repeat genotype sequencing system facilitates a more computationally efficient approach and obviates some or all extra assays for tandem-repeat genotyping.

As illustrated by the foregoing discussion, the present disclosure utilizes a variety of terms to describe features and advantages of the tandem-repeat genotype sequencing system. As used herein, for example, the term “methylation sequencing assay” refers to an assay that detects, measures, or quantifies methylation of cytosine from an oligonucleotide or other nucleotide sequence. In some cases, a methylation sequencing assay detects or quantifies methylation of cytosine at particular target genomic regions or in particular cell types. Some methylation sequencing assays quantify methylation in terms of methylation-level values.

As further used herein, the term “nucleotide read” (or simply “read”) refers to an inferred sequence of one or more nucleobases (or nucleobase pairs) from all or part of a sample nucleotide sequence (e.g., a sample genomic sequence, complementary DNA). In particular, a nucleotide read includes a determined or predicted sequence of nucleobase calls for a nucleotide sequence (or group of monoclonal nucleotide sequences) from a sample library fragment corresponding to a genomic sample. For example, in some cases, a sequencing device determines a nucleotide read by generating nucleobase calls for nucleobases passed through a nanopore of a nucleotide-sample slide, determined via fluorescent tagging, or determined from a cluster in a flow cell.

Also, as used herein, the term “genomic sample” refers to a target genome or portion of a genome undergoing sequencing. For example, a sample genome includes a sequence of nucleotides isolated or extracted from a sample organism (or a copy of such an isolated or extracted sequence). In particular, a sample genome includes a full genome that is isolated or extracted (in whole or in part) from a sample organism and composed of nitrogenous heterocyclic bases. A sample genome can include a segment of deoxyribonucleic acid (DNA), ribonucleic acid (RNA), or other polymeric forms of nucleic acids or chimeric or hybrid forms of nucleic acids noted below. In some cases, the sample genome is found in a sample prepared or isolated by a kit and received by a sequencing device.

Relatedly, the term “spanning nucleotide read” (or simply “spanning read”) refers to a nucleotide read that covers or encompasses a tandem-repeat region. In particular, a spanning nucleotide read covers an entire STR region, VNTR region, or other tandem-repeat region. For example, a spanning nucleotide read may include one or more flanking regions on both sides of a short tandem repeat region. The flanking regions may be of differing lengths.

As further used herein, the term “tandem repeat” refers to a motif or pattern of one or more nucleotides in DNA or RNA that is repeated consecutively one motif or pattern of nucleotides after another. A tandem repeat can include minisatellites in which 10 to 60 nucleotides are repeated as part of a pattern. By contrast, a tandem repeat can also include microsatellites or short tandem repeats in which less than ten nucleotides are repeated as part of a pattern. To illustrate, an example tandem repeat includes a sequence of TAAGC TAAGC TAAGC in which the sequence TAAGC is repeated three times. To further illustrate, a tandem repeat may also include dinucleotide repeats (e.g., GCGCGCGC) and trinucleotide repeats (e.g., CAGCAGCAGCAG).

As used herein, the term “short tandem repeat” or “STR” refers to a sequence of less than ten nucleotides that are repeated at least once. In particular, a short tandem repeat comprises a microsatellite with a nucleotide repeat unit, or motif, of one to seven base pairs in length. In this disclosure, the terms “short tandem repeat” and “microsatellite” are synonyms and can be used interchangeably. The nucleotide repeat units within an STR are identical and directly adjacent to each other. For example, an STR may be represented by an encoded nucleotide sequence such as CGG CGG CGG comprising three tandemly repeated CGG sequences.

Relatedly, the term “variable number tandem repeat” or “VNTR” refers to a sequence of DNA at a genomic region comprising a tandem repeat and for which a population of genomic samples exhibit variation. In some cases, a population exhibits variations in length of nucleotide repeat units at a particular VNTR region. Accordingly, a VNTR can act as an inherited allele.

As related to tandem repeats, the term “nucleotide repeat unit” (or simply “repeat unit”) refers to a single motif or unit of nucleotides within a pattern of nucleic acids that occur in multiple copies. In particular, a nucleotide repeat unit refers to a sequence of nucleic acids arranged next to at least one other identical sequence within a microsatellite, a minisatellite, or other tandem repeat. For example, a nucleotide repeat unit may be represented by an encoded nucleotide sequence, such as CGG or ATTCG.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

As used herein, the term “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1:1234570-1234870). In various implementations, a genomic coordinate includes a position within a reference genome. In some cases, a genomic coordinate is specific to a particular reference genome.

Relatedly, as used herein, the term “tandem-repeat region” refers to a genomic region comprising a tandem-repeat and surrounding or flanking nucleotide sequences. In particular, a tandem-repeat region includes an STR region, VNTR region, or other tandem-repeat and surrounding or flanking nucleotide sequences within a threshold number of nucleobases. Such a threshold number of nucleobases may, for instance, be 1,000 nucleobases on each side of the tandem repeat.

As used herein, the term “tandem-repeat allele” refers to a version or alternative form of tandem repeat region or tandem repeat nucleotide sequence. In some cases, a tandem-repeat allele is represented as a digital nucleotide sequence. For instance, a tandem-repeat allele may be represented by an encoded nucleotide sequence, such as by single-letter codes representing individual nucleobases (e.g., A, C, T, G), corresponding to particular genomic coordinates or tandem-repeat locus. More specifically, a tandem-repeat allele may be represented using a number of nucleotide repeat units. For example, a tandem-repeat allele may comprise any number of nucleotide repeat units (e.g., three, four, eight, etc.).

As used herein, the term “tandem-repeat genotype” refers to a determination or prediction of a particular genotype of a tandem-repeat region of a genomic sample. In particular, a tandem-repeat genotype can include a prediction of a particular genotype at an STR locus of a sample genome. In this disclosure, the tandem-repeat genotype sequencing system generates tandem-repeat genotypes comprising tandem-repeat alleles at tandem-repeat loci.

As used herein, the term “stutter model” refers to an algorithm or model for estimating the effects of stutter artifacts originating from PCR amplification on nucleotide reads. In particular, a stutter model predicts expected genotype probabilities of STR genotypes given the effects of stutter artifacts in STR regions. For example, a stutter model may comprise an algorithm or model that generates expected genotype probabilities of STR genotypes based on differing numbers of nucleotide repeat units in spanning nucleotide reads.

Relatedly, as used herein, the term “parameter” refers to a characteristic whose value affects a related state. In particular, the term parameter refers to a mathematical relationship or variable that affects the output of the stutter model. For example, parameters of the stutter model may comprise an increased-repeat-unit probability, a decreased-repeat-unit probability, a step size of a geometric distribution, and other values.

As used herein, the term “expected genotype probability” refers to the probability of a given STR genotype. In particular, expected genotype probability refers to a likelihood of a candidate STR genotype given differing numbers of nucleotide repeat units in spanning nucleotide reads. For example, a stutter model may generate an expected genotype probability given a distribution of spanning nucleotide reads having different numbers of nucleotide repeat units. An expected genotype probability may comprise a numerical value (e.g., 0-1) representing the probability of a given STR genotype.

As used herein, the term “candidate tandem-repeat genotype” refers to a potential or proposed tandem-repeat genotype based on nucleotide reads from a genomic sample corresponding to a tandem-repeat region. In particular, a candidate tandem-repeat genotype includes a potential or proposed tandem-repeat genotype for a particular locus of a genomic sample. In some cases, a candidate tandem-repeat genotype is identified based on spanning nucleotide reads for a genomic sample. As suggested above, in some embodiments, the tandem-repeat genotype sequencing system utilizes an expectation-maximization (EM) algorithm to determine expected genotype probabilities for candidate tandem-repeat genotypes.

As used herein, the term “converged genotype probabilities” refers to genotype probabilities that have settled to within an error range around other genotype probabilities. In particular, the term “converged genotype probabilities” refers to STR genotype probabilities from successive iterations whose difference fall within a threshold convergence range. For example, the tandem-repeat genotype sequencing system may utilize the stutter algorithm to generate expected genotype probabilities until the product of expected genotype probabilities in successive iteration fall within a threshold convergence range.

As also used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequence determined as representative of an organism. For example, a linear human reference genome may be GRCh38 (or other versions of reference genomes) from the Genome Reference Consortium.

As further used herein, the term “genotype call” refers to a determination or prediction of a particular genotype of a genomic sample at a genomic locus. In particular, a genotype call can include a prediction of a particular genotype of a genomic sample with respect to a reference genome or a reference sequence (e.g., STR-allele-reference sequence, VNTR-allele-reference sequence, minisatellite-allele-reference sequence) at a genomic coordinate or a genomic region. A genotype call is often determined for a genomic coordinate or genomic region at which an SNP or other variant has been identified for a population of organisms. In this disclosure, among other genotype calls, the tandem-repeat genotype sequencing system predicts genotype calls for tandem-repeat regions within a genomic sample (e.g., STR or microsatellite regions, minisatellite regions, VNTR regions, or guanine-quadruplex regions).

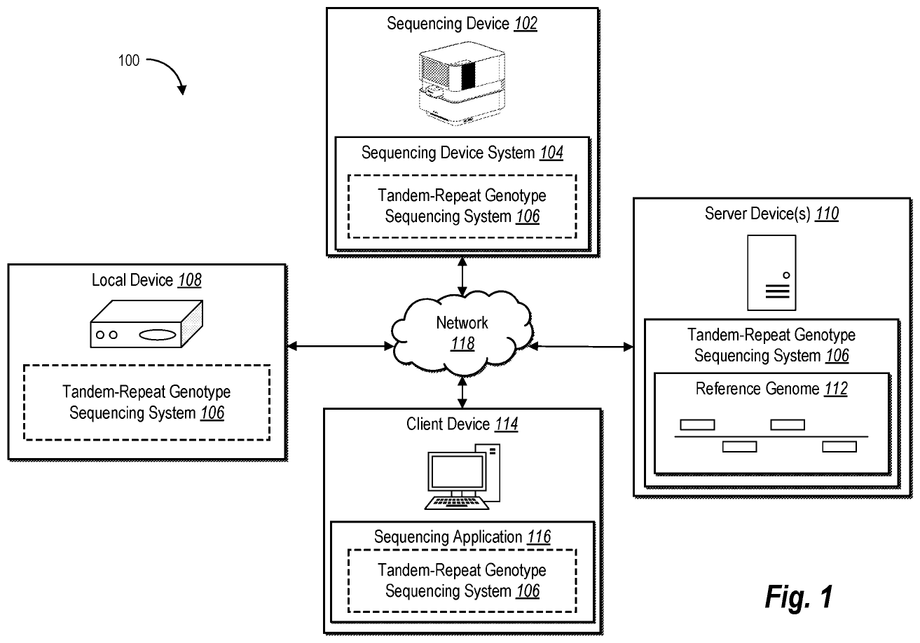

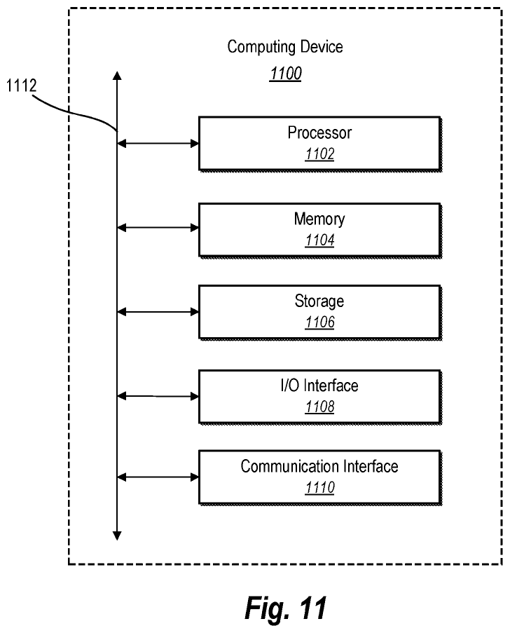

The following paragraphs describe the tandem-repeat genotype sequencing system with respect to illustrative figures that portray example embodiments and implementations. For example, FIG. 1 illustrates a schematic diagram of a computing system 100 in which a tandem-repeat genotype sequencing system 106 operates in accordance with one or more embodiments of the present disclosure. As illustrated, the computing system 100 includes a sequencing device 102 connected to a local device 108 (e.g., a local server device), one or more server device(s) 110, and a client device 114. As shown in FIG. 1, the sequencing device 102, the local device 108, the server device(s) 110, and the client device 114 can communicate with each other via a network 118. The network 118 comprises any suitable network over which computing devices can communicate. Example networks are discussed in additional detail below with respect to FIG. 11. While FIG. 1 shows an embodiment of the tandem-repeat genotype sequencing system 106, this disclosure describes alternative embodiments and configurations below.

As indicated by FIG. 1, the sequencing device 102 comprises a sequencing device system 104 for sequencing a genomic sample or other nucleic-acid polymer. In some examples, the sequencing device system 104 sequences oligonucleotides extracted from a genomic sample as part of a methylation sequencing assay. In some embodiments, by executing the sequencing device system 104 using a processor, the sequencing device 102 analyzes nucleotide fragments or oligonucleotides extracted from genomic samples to generate nucleotide reads or other data utilizing computer implemented methods and systems either directly or indirectly on the sequencing device 102. More particularly, the sequencing device 102 receives nucleotide-sample slides (e.g., flow cells) comprising nucleotide fragments extracted from samples and further copies and determines the nucleobase sequence of such extracted nucleotide fragments. For instance, the sequencing device 102 may determine nucleobase calls for nucleotide reads comprising CpG or other cytosine sites.

In one or more embodiments, the sequencing device 102 utilizes SBS to sequence nucleotide fragments into nucleotide reads and determine nucleobase calls for the nucleotide reads. As suggested above, by executing the sequencing device system 104, the sequencing device 102 can run one or more sequencing cycles as part of a sequencing run for a methylation sequencing assay. By executing the tandem-repeat genotype sequencing system 106, for instance, the sequencing device 102 can (i) sequence certain uracil bases that were converted from methylated cytosine bases and that are part of a nucleotide read and (ii) determine nucleobase calls of thymidine for such uracil bases as part of a methylation sequencing assay. In one or more embodiments, the sequencing device 102 utilizes Sequencing by Synthesis (SBS) to sequence nucleic-acid polymers into nucleotide reads.

As just suggested, in some embodiments, the tandem-repeat genotype sequencing system 106 can identify when a methyl or hydroxymethyl group has been added to a cytosine base of a genomic sample's deoxyribonucleic acid (DNA)—where the methylated cytosine base is often part of a cytosine-guanine-dinucleotide pair in a 5′-C-phosphate-G-3′ (CpG) configuration in mammals. For example, the tandem-repeat genotype sequencing system 106 can detect methylated cytosines by (i) enzymatically converting methylated or unmethylated cytosine bases at CpG or other sites from a sample nucleotide fragment into uracil bases (e.g., dihydrouracil); (ii) determining nucleobase calls of nucleotide reads for the genomic sample using the sequencing device 102, where the sequencing device 102 detects the uracil bases as thymidine bases during polymerase chain reaction (PCR) amplification; and (iii) comparing the nucleobase calls from the nucleotide reads to a reference genome or non-enzymatically converted nucleotide reads from the genomic sample. Based on the comparison of nucleotide reads from the sample to a reference genome or the non-enzymatically converted nucleotide reads, the tandem-repeat genotype sequencing system 106 can identify thymidine bases from the nucleotide reads that do not match cytosine bases at CpG or other sites within the reference genome or the non-enzymatically converted nucleotide reads and thereby detect methylated cytosine bases in a sample nucleotide fragment.

To convert cytosine to uracil, in some cases, the tandem-repeat genotype sequencing system 106 uses bisulfite or a non-bisulfite enzyme as part of a methylation sequencing assay. For instance, Tet-assisted pyridine borane sequencing (TAPS) uses a ten-eleven translocation (TET) enzyme for a methylation assay, as described by Yibin Liu et al., “Bisulfite-free Direct Detection of 5-Methylcystosine and 5-Hydroxymethylcystosine at Base Resolution,” 36 Nature Biotechnology 424-29 (2019). In some assays that rely on a TET enzyme, the tandem-repeat genotype sequencing system 106 executes a methylation sequencing assay that converts 5-Methylcystosine (5mC) and 5-Hydroxymethylcystosine (5hmC) into oxidized products using a TET enzyme and then uses an Apolipoprotein B mRNA Editing Enzyme, Catalytic Polypeptide (APOBEC) 3A or other APOBEC protein to deaminate unmodified cytosines by converting them to uracil bases.

In addition or in the alternative to communicating across the network 118, in some embodiments, the sequencing device 102 bypasses the network 118 and communicates directly with the local device 108 or the client device 114. By executing the sequencing device system 104, the sequencing device 102 can further store the nucleobase calls as part of base-call data that is formatted as a binary base call (BCL) file and send the BCL file to the local device 108 and/or the server device(s) 110.

As further indicated by FIG. 1, the local device 108 is located at or near a same physical location of the sequencing device 102. Indeed, in some embodiments, the local device 108 and the sequencing device 102 are integrated into a same computing device. The local device 108 may run the tandem-repeat genotype sequencing system 106 to generate, receive, analyze, store, and transmit digital data, such as by receiving base-call data or determining variant calls based on analyzing such base-call data. As shown in FIG. 1, the sequencing device 102 may send (and the local device 108 may receive) base-call data generated during a sequencing run of the sequencing device 102. By executing software in the form of the tandem-repeat genotype sequencing system 106, the local device 108 may align nucleotide reads with a reference genome 112 and determine genetic variants based on the aligned nucleotide reads. The local device 108 may also communicate with the client device 114. In particular, the local device 108 can send data to the client device 114, including a variant call file (VCF), methylation data, or other information indicating nucleobase calls, methylated cytosines, sequencing metrics, error data, STR genotypes, or other metrics.

As further indicated by FIG. 1, the server device(s) 110 are located remotely from the local device 108 and the sequencing device 102. Similar to the local device 108, in some embodiments, the server device(s) 110 include a version of the tandem-repeat genotype sequencing system 106. Accordingly, the server device(s) 110 may generate, receive, analyze, store, and transmit digital data, such as by receiving base-call and/or methylation data or determining variant calls or genotype calls for tandem-repeat alleles based on analyzing such base-call data. As indicated above, the sequencing device 102 may send (and the server device(s) 110 may receive) base-call data and/or methylation data from the sequencing device 102. The server device(s) 110 may also communicate with the client device 114. In particular, the server device(s) 110 can send data to the client device 114, including VCFs, methylation data, tandem-repeat genotypes, or other sequencing related information.

In some embodiments, the server device(s) 110 comprise a distributed collection of servers where the server device(s) 110 include a number of server devices distributed across the network 118 and located in the same or different physical locations. Further, the server device(s) 110 can comprise a content server, an application server, a communication server, a web-hosting server, or another type of server.

As indicated above, as part of the server device(s) 110 or the local device 108, the tandem-repeat genotype sequencing system 106 can accurately predict tandem-repeat genotypes from a genomic sample by utilizing a stutter model to analyze spanning reads for a genomic sample. For instance, the tandem-repeat genotype sequencing system 106 identifies spanning reads for a genomic sample that cover tandem-repeat region. The tandem-repeat genotype sequencing system 106 can utilize a stutter model to determine expected genotype probabilities and updates parameters of the stutter model based on the expected genotype probabilities. The tandem-repeat genotype sequencing system 106 can iteratively perform the above-described expectation and maximization stages until reaching converged genotype probabilities of tandem-repeat genotypes and determining a genotype call for the tandem-repeat region based on the converged genotype probabilities.

As further illustrated and indicated in FIG. 1, by executing a sequencing application 116, the client device 114 can generate, store, receive, and send digital data. In particular, the client device 114 can receive sequencing data from the local device 108 or receive call files (e.g., BCL) and sequencing metrics from the sequencing device 102. For example, the client device 114 can receive methylation data from the local device 108. Furthermore, the client device 114 may communicate with the local device 108 or the server device(s) 110 to receive a VCF, methylation report file, tandem-repeat genotyping file, or other metric files comprising nucleobase calls, methylation data, genotype calls, and/or other metrics, such as a base-call-quality metrics or pass-filter metrics. The client device 114 can accordingly present or display information pertaining to genotype calls, methylation data, variant calls, or other nucleobase calls within a graphical user interface of the sequencing application 116 to a user associated with the client device 114. For example, the client device 114 can present genotype calls for tandem-repeat regions and/or sequencing metrics for a sequenced genomic sample within a graphical user interface of the sequencing application 116.

Although FIG. 1 depicts the client device 114 as a desktop or laptop computer, the client device 114 may comprise various types of client devices. For example, in some embodiments, the client device 114 includes non-mobile devices, such as desktop computers or servers, or other types of client devices. In yet other embodiments, the client device 114 includes mobile devices, such as laptops, tablets, mobile telephones, or smartphones. Additional details regarding the client device 114 are discussed below with respect to FIG. 11.

As further illustrated in FIG. 1, the client device 114 includes the sequencing application 116. The sequencing application 116 may be a web application or a native application stored and executed on the client device 114 (e.g., a mobile application, desktop application). The sequencing application 116 can include instructions that (when executed) cause the client device 114 to receive data from the tandem-repeat genotype sequencing system 106 and present, for display at the client device 114, base-call data or data from a VCF, tandem-repeat report file, tandem-repeat metrics file, or data from a methylation sequencing assay.

As further illustrated in FIG. 1, a version of the tandem-repeat genotype sequencing system 106 may be located and implemented (e.g., entirely or in part) on the client device 114 or the sequencing device 102. In yet other embodiments, the tandem-repeat genotype sequencing system 106 is implemented by one or more other components of the computing system 100, such as the local device 108. In particular, the tandem-repeat genotype sequencing system 106 can be implemented in a variety of different ways across the sequencing device 102, the local device 108, the server device(s) 110, and the client device 114. For example, the tandem-repeat genotype sequencing system 106 can be downloaded from the server device(s) 110 to the client device 114, the sequencing device 102, and/or the local device 108 where all or part of the functionality of the tandem-repeat genotype sequencing system 106 is performed at each respective device within the computing system 100.

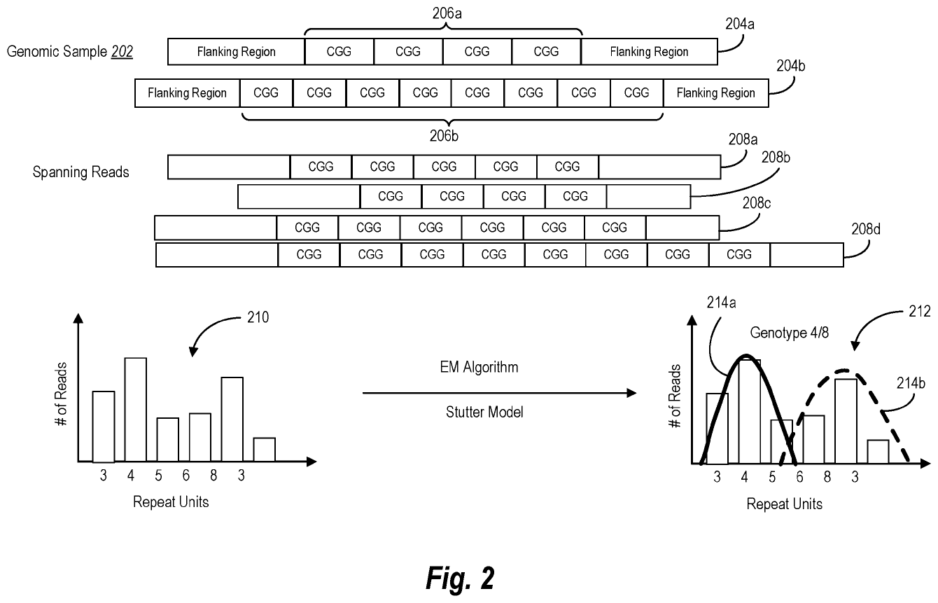

As indicated above, the tandem-repeat genotype sequencing system 106 can genotype one or more tandem-repeat regions from a genomic sample by utilizing an Expectation-Maximization (EM) algorithm and a stutter model. FIG. 2 illustrates an overview of the EM algorithm intuition utilized by the tandem-repeat genotype sequencing system 106 in accordance with one or more embodiments of the present disclosure.

FIG. 2 illustrates a genomic sample 202 comprising alleles 204a and 204b. The alleles 204a-204b comprise or correspond to tandem-repeat regions 206a-206b, respectively, located within the genomic sample 202. The tandem-repeat regions 206a-206b are surrounded by flanking regions. As illustrated, the tandem-repeat region 206a for the allele 204a comprises four nucleotide repeat units. By contrast, the tandem-repeat region 206b for the allele 204b comprises eight nucleotide repeat units. As mentioned, many existing methylation sequencing systems accurately predict genotypes for tandem-repeat regions using PCR-free whole genome sequencing data. However, some assays, including methylation sequencing assays, require a PCR step. PCR steps may, for example through enzyme slippage events, wrongfully amplify tandem-repeat regions (e.g., the tandem-repeat regions 206a-206b). The tandem-repeat genotype sequencing system 106 improves tandem-repeat genotyping accuracy even when utilizing data from PCR-reliant assays.

Because of PCR amplification errors, nucleotide reads sequenced for the genomic sample 202 include differing numbers of nucleotide repeat units. The tandem-repeat genotype sequencing system 106 extracts or identifies spanning nucleotide reads 208a, 208b, 208c, and 208d from among nucleotide reads of the genomic sample 202. As illustrated in FIG. 2, the spanning nucleotide reads 208a-208d comprise nucleotide reads that cover an entire tandem-repeat region and flanking regions outside of the tandem-repeat region. The spanning nucleotide reads 208a-208d comprise five, four, six, and eight nucleotide repeat units, respectively.

As just indicated, the spanning nucleotide reads 208a-208d depicted in FIG. 2 comprise a subset of sample nucleotide reads from nucleotide reads sequenced for the genomic sample 202. Graph 210 depicts a distribution of all spanning nucleotide reads for a tandem-repeat region. The x axis of the graph 210 represents the number of nucleotide repeat units within a spanning read, and the y axis of the graph 210 represents the number of spanning reads having the corresponding number of nucleotide repeat units. Because of slippage events, therefore, some of the spanning nucleotide reads 208a-208d do not match either the allele 204a or the allele 204b of the genomic sample 202.

The tandem-repeat genotype sequencing system 106 leverages differing numbers of nucleotide repeat units in the spanning nucleotide reads 208a-208d to accurately predict a tandem-repeat genotype for the genomic sample 202. More specifically, the tandem-repeat genotype sequencing system 106 determines candidate tandem-repeat genotypes given the differing numbers of nucleotide repeat units in the spanning nucleotide reads 208a-208d. The tandem-repeat genotype sequencing system 106 may utilize an EM algorithm to estimate a genotype call from the finite candidate tandem-repeat genotypes.

Generally, the tandem-repeat genotype sequencing system 106 utilizes an EM algorithm and a stutter model to cluster the spanning nucleotide reads into different groups based on the numbers of nucleotide repeat units. As illustrated by graph 212 in FIG. 2, for instance, the tandem-repeat genotype sequencing system 106 clusters the spanning nucleotide reads into two groups-group 214a and group 214b. Each of the groups 214a-214b correspond to one of the alleles 204a-204b from the genomic sample 202. The tandem-repeat genotype sequencing system 106 determines that the number of nucleotide repeat units at the center of each group most probably comprises the true number of repeat units found in the alleles 204a-204b. For example, as depicted in FIG. 2, the tandem-repeat genotype sequencing system 106 predicts, based on the group 214a, that a first allele comprises four nucleotide repeat units and a second allele comprises eight nucleotide repeat units.

In some implementations, the tandem-repeat genotype sequencing system 106 utilizes an EM algorithm to estimate a genotype call for the genomic sample 202. For example, during an expectation phase of the EM algorithm, the tandem-repeat genotype sequencing system 106 assigns each data point to a cluster or group. For example, the tandem-repeat genotype sequencing system 106 can assign a number of nucleotide repeat units to one of the group 214a or the group 214b. As mentioned, the group 214a represents a first allele and the group 214b represents a second allele from the genomic sample 202. During a maximization phase of the EM algorithm, the tandem-repeat genotype sequencing system 106 updates the parameters for each group based on the number of nucleotide repeat units within the group. The tandem-repeat genotype sequencing system 106 may determine genotype probabilities based on the clustered spanning reads. As mentioned, the tandem-repeat genotype sequencing system 106 may iteratively repeat the phases within the EM algorithm until reaching converged genotype probabilities. The tandem-repeat genotype sequencing system 106 may determine tandem-repeat alleles corresponding with the highest allele probabilities comprise the tandem-repeat genotypes.

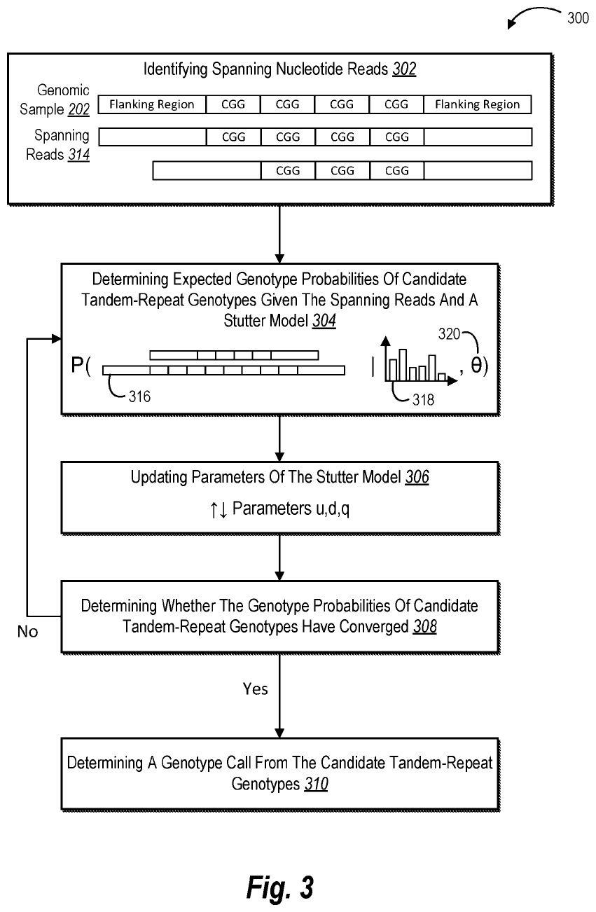

FIG. 3 depicts an overview the tandem-repeat genotype sequencing system 106 determining a genotype call for a tandem-repeat region in accordance with one or more implementations of the present disclosure. By way of overview, FIG. 3 illustrates a series of acts 300 comprising an act 302 of identifying spanning nucleotide reads for a genomic sample 312, an act 304 of determining expected genotype probabilities of candidate tandem-repeat genotypes given the spanning nucleotide reads and a stutter model, an act 306 of updating parameters of the stutter model, an act 308 of determining whether the genotype probabilities of candidate tandem-repeat genotypes have converged, and an act 310 of determining a genotype call from the candidate tandem-repeat genotypes.

As shown in FIG. 3, the series of acts 300 includes the act 302 of identifying spanning nucleotide reads for the genomic sample 312. In particular, the tandem-repeat genotype sequencing system 106 identifies, from nucleotide reads sequenced for the genomic sample 312, spanning nucleotide reads that cover a tandem-repeat region. In some embodiments, for example, the tandem-repeat genotype sequencing system 106 uses the sequencing device 102 to perform sequencing runs for a methylation sequencing assay to determine the nucleotide reads for the genomic sample 312 from which the spanning nucleotide reads are identified. Alternatively, in certain cases, the tandem-repeat genotype sequencing system 106 receives data representing the nucleotide reads generated by the methylation sequencing assay for the genomic sample 312 and identifies the spanning nucleotide reads from among the nucleotide reads based on a comparison of the nucleotide reads with a reference genome.

As shown, FIG. 3 illustrates a genomic sample 312 comprising a tandem-repeat region comprising four nucleotide repeat units, where each nucleotide repeat unit comprises CGG. The tandem-repeat genotype sequencing system 106 accesses nucleotide reads sequenced for the genomic sample 312 that cover the tandem-repeat region. The tandem-repeat genotype sequencing system 106 selects, from the nucleotide reads, spanning nucleotide reads 314 that include entire repeat sequences bounded by flanking regions. The spanning nucleotide reads 314 may contain differing numbers of nucleotide repeat units. For example, the spanning nucleotide reads 314 illustrated in FIG. 3 comprise four and three nucleotide repeat units. While FIG. 3 illustrates merely two spanning nucleotide reads for space considerations, the tandem-repeat genotype sequencing system 106 extracts any number of spanning nucleotide reads from the nucleotide reads of the genomic sample 312.

As further illustrated in FIG. 3, the tandem-repeat genotype sequencing system 106 performs the act 304 of determining expected genotype probabilities of candidate tandem-repeat genotypes given the spanning nucleotide reads and a stutter model. Generally, the tandem-repeat genotype sequencing system 106 determines a distribution 318 of spanning reads having different numbers of nucleotide repeat units. As illustrated in FIG. 3, the x axis of the distribution 318 comprises differing numbers of repeat units, and the y axis comprises numbers of spanning nucleotide reads with those differing numbers of repeat units. The tandem-repeat genotype sequencing system 106 utilizes a stutter model 320 that captures PCR amplification errors given the distribution 318. As further shown in FIG. 3, the tandem-repeat genotype sequencing system 106 determines probabilities of a candidate genotype 316 based on the distribution 318 and the stutter model 320.

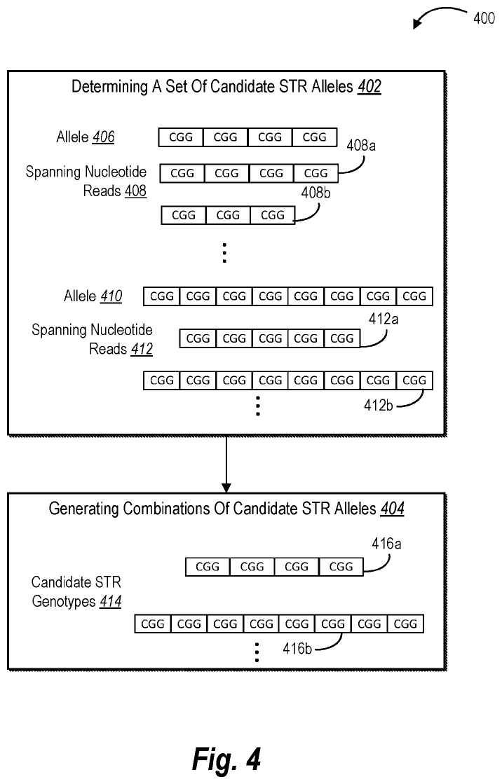

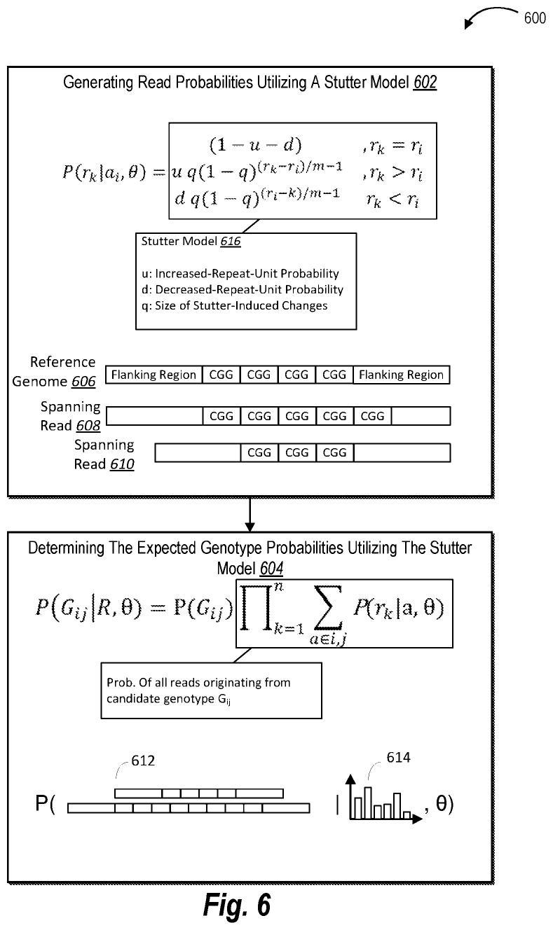

In some implementations, the act 304 comprises an expectation stage of an expectation maximization (EM) algorithm. FIG. 4 and the corresponding discussion further detail the tandem-repeat genotype sequencing system 106 determining candidate tandem-repeat alleles and genotypes in accordance with one or more embodiments of the present disclosure as a precursor to an expectation stage. FIG. 6 and the corresponding discussion describe the tandem-repeat genotype sequencing system 106 determining the expected genotype probability as part of an expectation stage in accordance with one or more embodiments.

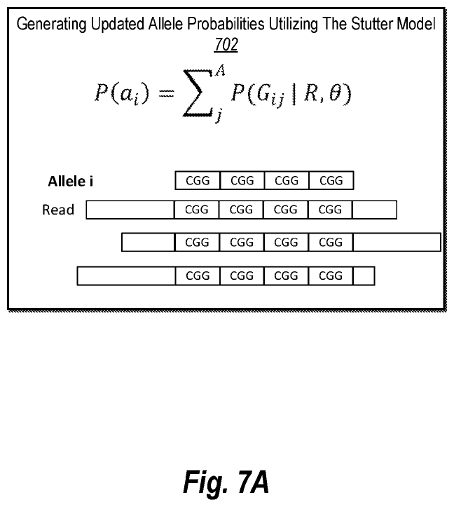

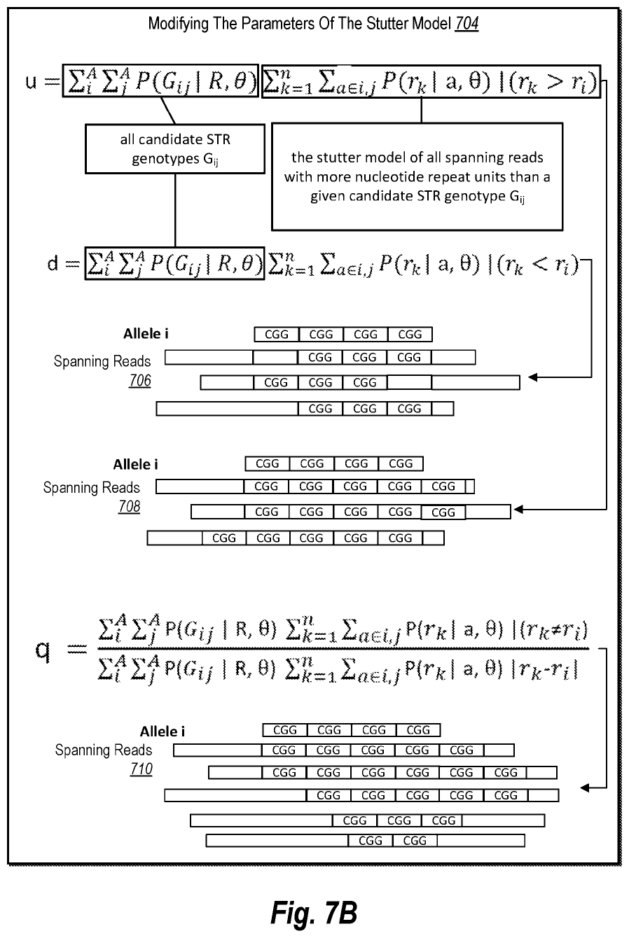

FIG. 3 further illustrates the tandem-repeat genotype sequencing system 106 performing the act 306 of updating parameters of the stutter model. Generally, the stutter model 320 comprises the following parameters: (u) an increased-repeat-unit probability of a given nucleotide read comprising more repeat units than a reference genome within a corresponding tandem-repeat region, (d) a decreased-repeat-unit probability of the given nucleotide read comprising fewer nucleotide repeat units than the reference genome within a corresponding tandem-repeat region, and (q) a size of stutter-induced changes in the spanning nucleotide reads. The tandem-repeat genotype sequencing system 106 modifies the parameters of the stutter model 320 based on the expected genotype probabilities. In some implementations, the act 306 comprises a maximization stage of an EM algorithm. FIGS. 7A-7B illustrate the tandem-repeat genotype sequencing system 106 modifying the parameters of the stutter model as part of a maximization stage in accordance with one or more embodiments.

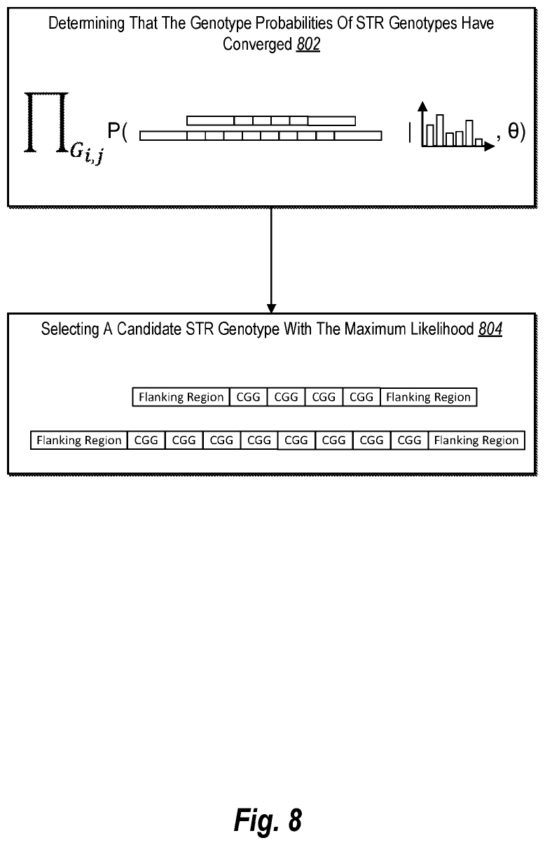

As further illustrated in FIG. 3, the tandem-repeat genotype sequencing system 106 performs the act 308 of determining whether the genotype probabilities of candidate tandem-repeat genotypes have converged. To determine convergence, the tandem-repeat genotype sequencing system 106 compares expected genotype probabilities from successive iterations of utilizing the stutter model 320. In some implementations, the tandem-repeat genotype sequencing system 106 determines that the genotype probabilities of candidate tandem-repeat genotypes have converged when the differences between the expected tandem-repeat genotypes fall within a threshold value. For example, the tandem-repeat genotype sequencing system 106 may determine whether the product of expected tandem-repeat genotype probabilities in successive iterations fall within a threshold convergence range. FIG. 8 provides additional detail regarding how the tandem-repeat genotype sequencing system 106 determines that genotype probabilities of candidate tandem-repeat genotypes have converged in accordance with one or more embodiments.

If the tandem-repeat genotype sequencing system 106 determines that the genotype probabilities of candidate tandem-repeat genotypes have not converged, as indicated by FIG. 3, the tandem-repeat genotype sequencing system 106 repeats the act 304 and the act 306 to generate additional expected genotype probabilities of candidate tandem-repeat genotypes using adjusted parameters. In some examples, the tandem-repeat genotype sequencing system 106 iteratively repeats the act 304 and the act 306, or the expectation and maximization stages of an EM algorithm, respectively, until the tandem-repeat genotype sequencing system 106 determines that the genotype probabilities of candidate tandem-repeat genotypes have converged. In some implementations, based on the tandem-repeat genotype sequencing system 106 determining that the genotype probabilities of candidate tandem-repeat genotypes have converged, the tandem-repeat genotype sequencing system 106 proceeds to perform the act 310 of determining a genotype call from the candidate tandem-repeat genotypes.

As illustrated in FIG. 3, the tandem-repeat genotype sequencing system 106 performs the act 310 of determining a genotype call from the candidate tandem-repeat genotypes. As part of the act 310, the tandem-repeat genotype sequencing system 106 identifies a tandem-repeat genotype having the highest likelihood or probability of being the true genotype of the genomic sample. In some cases, the tandem-repeat genotype sequencing system 106 performs the act 310 by identifying the candidate tandem-repeat genotypes having the highest genotype probabilities.

As just described, FIG. 3 shows the tandem-repeat genotype sequencing system 106 determining genotype calls for a tandem-repeat region within a genomic sample. As depicted in FIGS. 4-8, this disclosure generally depicts and describes (i) determining candidate STR alleles from spanning nucleotide reads covering an STR region of a genomic sample, initializing values for an EM algorithm and a stutter model, (iii) utilizing the EM algorithm and the stutter model to iteratively predict STR genotype probabilities and update the stutter model's parameters based on spanning reads, and (iv) generating a genotype call from candidate STR genotypes that the genomic sample comprises one or more STR alleles at the STR region based on converged genotype probabilities. While FIGS. 4-8 use an STR region, STR genotypes, and STR alleles as examples, the tandem-repeat genotype sequencing system 106 can perform the same operations for other tandem-repeat regions, tandem-repeat genotypes, and tandem-repeat alleles.

As mentioned, in some embodiments, the tandem-repeat genotype sequencing system 106 determines expected genotypes for candidate STR alleles. FIG. 4 illustrates the tandem-repeat genotype sequencing system 106 generating candidate STR genotypes in accordance with one or more embodiments of the present disclosure. Generally, the tandem-repeat genotype sequencing system 106 identifies candidate STR alleles based on the nucleotide repeat units exhibited by spanning nucleotide reads and determines candidate STR genotypes based on combinations of the STR alleles. By way of overview, FIG. 4 illustrates a series of acts 400 comprising an act 402 of determining a set of candidate STR alleles and an act 404 of generating combinations of candidate STR alleles.

The tandem-repeat genotype sequencing system 106 efficiently predicts genotypes for STR regions of a sample genome by predicting genotype probabilities for select candidate STR genotypes. Instead of calculating probabilities for a nearly infinite number of possible STR genotypes, as indicated by FIG. 4, the tandem-repeat genotype sequencing system 106 determines candidate STR genotypes based on the spanning nucleotide reads.

As illustrated in FIG. 4, the tandem-repeat genotype sequencing system 106 performs the act 402 of determining a set of candidate STR alleles. In particular, the tandem-repeat genotype sequencing system 106 determines a set of candidate STR alleles based on differing numbers of nucleotide repeat units in the spanning nucleotide reads. In some implementations, the tandem-repeat genotype sequencing system 106 determines numbers of nucleotide repeat units in each spanning nucleotide read corresponding to an STR region within a reference genome. The tandem-repeat genotype sequencing system 106 determines candidate STR alleles having the numbers of repeat units present in individual spanning nucleotide reads.

For example, and as illustrated in FIG. 4, the tandem-repeat genotype sequencing system 106 identifies spanning nucleotide reads 408 and spanning nucleotide reads 412 that align with an STR region of a reference genome. The spanning nucleotide reads 408 originate from an allele 406 of the genomic sample, and the spanning nucleotide reads 412 originate from an allele 410 of the genomic sample. The allele 406 and the allele 410 comprise alleles at the same locus or genomic coordinates corresponding to an STR region in a reference genome. In some embodiments, because the number of nucleotide repeat units in the allele 406 and the allele 410 are unknown, the tandem-repeat genotype sequencing system 106 does not make a distinction between the spanning nucleotide reads 408 and the spanning nucleotide reads 412 beyond determining numbers of nucleotide repeat units and later identifying a count of such respective spanning nucleotide reads. Instead, the tandem-repeat genotype sequencing system 106 simply identifies all spanning nucleotide reads corresponding to an STR locus of a reference genome.

As part of performing the act 402, the tandem-repeat genotype sequencing system 106 determines numbers of nucleotide repeat units present in the spanning nucleotide reads. As shown, the spanning nucleotide reads 408 and the spanning nucleotide reads 412 comprise differing numbers of a nucleotide repeat unit (e.g., CGG). As illustrated, the spanning nucleotide read 408a comprises four repeat units, the spanning nucleotide read 408b comprises three repeat units, the spanning nucleotide read 412a comprises five repeat units, and the spanning nucleotide read 412b comprises eight repeat units. The tandem-repeat genotype sequencing system 106 determines candidate STR alleles based on the observed numbers of nucleotide repeat units from the spanning nucleotide reads 408 and the spanning nucleotide reads 412. More specifically, the tandem-repeat genotype sequencing system 106 generates candidate STR alleles having numbers of nucleotide repeat units found in the spanning nucleotide reads. For example, the tandem-repeat genotype sequencing system 106 determines a set of candidate STR alleles comprising four, three, five, and eight nucleotide repeat units based on the spanning nucleotide reads 408 and the spanning nucleotide reads 412 containing four, three, five, and eight nucleotide repeat units, respectively.

As further illustrated in FIG. 4, the tandem-repeat genotype sequencing system 106 performs the act 404 of generating combinations of candidate STR alleles. As described previously, a candidate STR genotype refers to a potential STR genotype comprising STR alleles for a particular locus of a genomic sample. As part of performing the act 404, the tandem-repeat genotype sequencing system 106 creates all possible combinations of two candidate STR alleles to create candidate STR genotypes 414. As illustrated in FIG. 4, the tandem-repeat genotype sequencing system 106 generates combinations of candidate alleles having four, three, five, and eight nucleotide repeat units. For example, the tandem-repeat genotype sequencing system 106 generates a candidate STR genotype comprising a first candidate STR allele having four nucleotide repeat units and a second candidate STR allele having four nucleotide repeat units. The tandem-repeat genotype sequencing system 106 generates various combinations of candidate STR alleles including a candidate STR allele 416a comprising four repeat units and a candidate STR allele 416b comprising eight nucleotide repeat units.

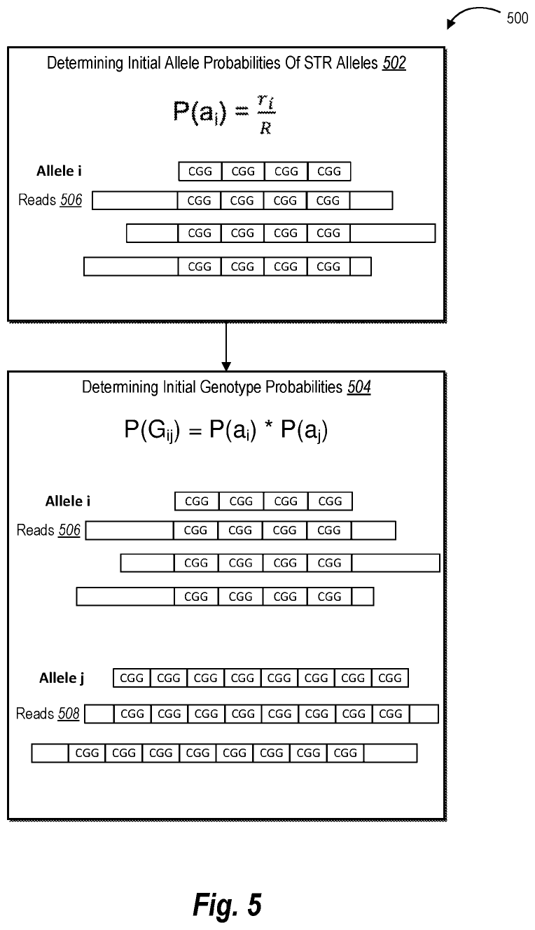

In some implementations, the tandem-repeat genotype sequencing system 106 generates initial genotype probabilities. FIG. 5 illustrates the tandem-repeat genotype sequencing system 106 determining initial genotype probabilities in accordance one or more embodiments of the present disclosure. Generally, the tandem-repeat genotype sequencing system 106 initializes STR genotype probabilities as a starting point for values within the EM algorithm. The tandem-repeat genotype sequencing system 106 determines initial allele probabilities and initial genotype probabilities based on observed STR alleles in the spanning nucleotide reads. By way of overview, FIG. 5 illustrates a series of acts 500 including an act 502 of determining initial allele probabilities of STR alleles, and an act 504 of determining initial genotype probabilities.

As shown in FIG. 5, the tandem-repeat genotype sequencing system 106 performs the act 502 of determining initial allele probabilities of STR alleles. More specifically, the tandem-repeat genotype sequencing system 106 initializes allele probabilities for candidate STR alleles. In some examples, the tandem-repeat genotype sequencing system 106 determines an initial allele probability for a given candidate STR allele based on a number of corresponding spanning nucleotide reads and a total number of spanning nucleotide reads. As suggested above and depicted in FIG. 5, spanning nucleotide reads have an equal number of nucleotide repeat units as the corresponding candidate STR allele.

For example, and as illustrated in FIG. 5, allele i represents a candidate STR allele having four nucleotide repeat units (e.g., four instances of a nucleotide repeat unit of CGG). The tandem-repeat genotype sequencing system 106 identifies, from the spanning nucleotide reads, corresponding spanning nucleotide reads 506 that have an equal number of nucleotide repeat units (e.g., four) as the candidate STR allele i. In some implementations, the tandem-repeat genotype sequencing system 106 determines the initial allele probability of an STR allele based on a number of spanning nucleotide reads supporting a particular allele and a total number of spanning reads. For instance, in some examples, the tandem-repeat genotype sequencing system 106 determines an initial allele probability of allele i by dividing a number of spanning nucleotide reads supporting a particular allele by a total number of spanning reads, as represented in the following equation:

P(aᵢ)=rᵢ/R

where P(aᵢ) represents the initial allele probability of allele i (aᵢ), rᵢ represents the number of corresponding spanning nucleotide reads, and R represents the total number of spanning nucleotide reads. In the example illustrated in FIG. 5, based on identifying three corresponding spanning nucleotide reads, the tandem-repeat genotype sequencing system 106 determines that rᵢ equals three. As suggested by FIG. 5, the tandem-repeat genotype sequencing system 106 similarly determines initial allele probabilities for other alleles j, k, or additional alleles using a similar equation for P(aⱼ)=rⱼ/R, P(aₖ)=rₖ/R, etc., depending on the candidate alleles exhibited by the spanning nucleotide reads (e.g., spanning nucleotide reads 508 for candidate STR allele j) for a genomic sample.

As further illustrated in FIG. 5, the tandem-repeat genotype sequencing system 106 performs the act 504 of determining initial genotype probabilities for candidate STR genotypes. As previously mentioned, the candidate STR genotypes comprise combinations of candidate STR alleles. Accordingly, the tandem-repeat genotype sequencing system 106 determines the initial genotype probabilities based on combinations of the initial allele probabilities of STR alleles.

FIG. 5 illustrates the tandem-repeat genotype sequencing system 106 determining an initial genotype probability for a candidate STR genotype comprising allele i and allele j. Allele i comprises four nucleotide repeat units, and allele j comprises eight nucleotide repeat units. The tandem-repeat genotype sequencing system 106 combines the initial allele probabilities for the allele i and the allele j. In some implementations, the tandem-repeat genotype sequencing system 106 determines an initial genotype probability of a candidate STR genotype using the following equation:

$$P ( {G}_{i j} ) = P ( {a}_{i} ) \times P ( {a}_{j} )$$

where P(Gᵢⱼ) represents the initial genotype probability of genotype ij (Gᵢⱼ), P(aᵢ) represents the initial allele probability of allele i, and P(aⱼ) represents the initial allele probability of allele j. Similarly, As suggested by FIG. 5, the tandem-repeat genotype sequencing system 106 determines genotype probabilities for other combinations of alleles i, j, k, or additional alleles using similar equations for P(Gⱼₖ)=P(aⱼ)×P(aₖ), P(Gⱼⱼ)=P(aⱼ)×P(aⱼ), P(Gₖₖ)=P(aₖ)×P(aₖ), P(Gᵢₖ)=P(aᵢ)×P(aₖ), etc., depending on the candidate alleles exhibited by the spanning nucleotide reads for a genomic sample.

As previously mentioned, the tandem-repeat genotype sequencing system 106 utilizes an EM algorithm to generate expected genotype probabilities utilizing a stutter model. FIG. 6 illustrates the tandem-repeat genotype sequencing system 106 performing an expectation stage of an EM algorithm and determining expected genotype probabilities in accordance with one or more embodiments of the present disclosure. By way of overview, FIG. 6 illustrates a series of acts 600 comprising an act 602 of generating read probabilities using a stutter model and, based on the differing numbers of nucleotide repeat units in the spanning nucleotide reads, an act 604 of determining the expected genotype probabilities utilizing the stutter model.

As shown in FIG. 6, the tandem-repeat genotype sequencing system 106 performs the act 602 of generating read probabilities utilizing a stutter model. FIG. 6 illustrates a stutter model 616 and parameters of the stutter model 616. The stutter model 616 captures PCR amplification errors introduced during PCR steps based on a distribution of spanning reads. As shown, the stutter model 616 comprises various parameters. Among others, parameters of the stutter model 616 include, (u) an increased-repeat-unit probability, (d) a decreased-repeat-unit probability, and (q) a size of stutter-induced changes. The following paragraphs describe each of these parameters in additional detail.

The parameter u equals an increased-repeat-unit probability of a given nucleotide read comprising more nucleotide repeat units than a reference genome within a corresponding STR region or other tandem-repeat region. Accordingly, the increased-repeat-unit probability parameter (u) represents the likelihood that a spanning nucleotide read originating from a candidate STR allele contains more repeat units than the tandem-repeat region of the reference genome 606. In other words, the increased-repeat-unit probability parameter (u) contains a probability that a stutter adds one or more repeat units from the true allele in an observed nucleotide read. For example, and as shown in FIG. 6, spanning read 608 comprises five nucleotide repeat units, which is one nucleotide repeat unit more than the four nucleotide repeat units in a reference genome 606. The increased-repeat-unit probability parameter (u), therefore, captures the likelihood that the spanning read 608 and other spanning reads containing any number of more nucleotide repeat units than the reference genome 606 originate from a candidate STR allele. As described below, FIG. 7B illustrates the tandem-repeat genotype sequencing system 106 adjusting the increased-repeat-unit probability (u) parameter in accordance with one or more embodiments.

The parameter d equals a decreased-repeat-unit probability of a given nucleotide read comprising fewer nucleotide repeat units than the reference genome within the corresponding STR region or other tandem-repeat region. Accordingly, the decreased-repeat-unit probability parameter (d) represents the likelihood that a spanning read originating from a candidate STR allele contains fewer repeat units than the reference genome 606. In particular, the decreased-repeat-unit probability parameter (d) contains the probability that stutter removes one or more nucleotide repeat units from the true allele in an observed nucleotide read. For example, and as shown in FIG. 6, a spanning read 610 comprises three nucleotide repeat units, which is one nucleotide repeat unit less than the four nucleotide repeat units in the reference genome 606. The decreased-repeat-unit probability parameter (d), therefore, captures the likelihood that the spanning read 610 and other spanning reads containing any number of fewer nucleotide repeat units than the reference genome 606 originate from a candidate STR allele. As explained below, FIG. 7B illustrates the tandem-repeat genotype sequencing system 106 adjusting the decreased-repeat-unit probability (d) parameter in accordance with one or more embodiments.

In some embodiments, the tandem-repeat genotype sequencing system 106 initializes values for the increased-repeat-unit probability (u) parameter and the decreased-repeat-unit probability (d) parameter. For example, the tandem-repeat genotype sequencing system 106 can assign random values to the increased-repeat-unit probability (u) and the decreased-repeat-unit probability (d) in a first iteration. In real world experiments, however, PCR amplification is more likely to miss or delete a repeat unit than add an extra repeat unit. Therefore, in some implementations, the tandem-repeat genotype sequencing system 106 initializes the stutter model with a lower increased-repeat-unit probability (u) value than the decreased-repeat-unit probability (d) value for a first iteration. More specifically, the tandem-repeat genotype sequencing system 106 initializes a value for the decreased-repeat-unit probability that exceeds an initialized value for the increased-repeat-unit probability.

As previously mentioned, the stutter model 616 also includes a parameter comprising a size of stutter-induced changes (q). For example, the size of stutter-induced changes (q) may indicate a difference between a number of nucleotide repeat units in a nucleotide read and a number of nucleotide repeat units in the reference allele of a reference genome. In some embodiments, the size of stutter-induced changes (q) indicates a difference between a number of nucleotide repeat units in nucleotide read and a number of nucleotide repeat units in the true allele of the genomic sample.

The tandem-repeat genotype sequencing system 106 may utilize the size of stutter-induced changes (q) to determine a constant mutation rate within a geometric distribution of spanning reads. In some implementations, the tandem-repeat genotype sequencing system 106 utilizes the size of stutter-induced changes (q) to determine a step size of a geometric distribution corresponding to the spanning nucleotide reads, where the step size is represented as 1-q. Generally, the geometric distribution describes the probability of observing a first nucleotide repeat unit at a locus after a certain number of mutations (e.g., a gain or loss of nucleotide repeat units) given a step size (1-q) representing a constant mutation rate. To illustrate, a small value q allows for frequent multi-step stutter artifacts, whereas larger q values near I restrict stutter artifacts to single-step changes.

As indicated by the formulas in FIG. 6, a geometric distribution for a mutation rate gives the probability that the first occurrence of success requires k independent trials, each with success probability p. If the probability of success on each trial equals p, then the probability that the kth trial (out of finite trials) constitutes the first success is U=0.01 or that 1% of reads have more nucleotide repeat units than a reference genome. Each nucleotide repeat unit within a nucleotide read may comprise an independent trial. An occurrence of success is defined by a repeat unit within a read matching with a repeat unit in a reference genome. A trial is considered a failure when a repeat unit within a read is different from the repeat unit within the reference genome. Trial failures can result from missing or additional repeat units within a nucleotide read relative to a reference genome.

For example, and as illustrated in FIG. 6, the spanning read 608 contains one failed trial comprising the rightmost repeat unit (e.g., rightmost CGG) that does not match up with the repeat units in the reference genome 606. The spanning read 608 also includes four success trials with the second rightmost repeat unit equaling a first occurrence of success. In another example depicted in FIG. 6, the spanning read 610 is missing the leftmost repeat unit present in the reference genome 606. This missing repeat unit comprises a single failed trial. The leftmost repeat unit within the spanning read 610 is the first repeat unit within the spanning read 610 that matches repeat units within the reference genome 606 and, accordingly, qualifies as a success trial. Based on the examples provided above, the size of stutter-induced changes (q) represents a probability of success on a first trial. The step size of geometric distribution (1-q) represents a mutation rate of a single repeat unit.

As shown in FIG. 6, the tandem-repeat genotype sequencing system 106 generates read probabilities utilizing the stutter model 616. More specifically, the tandem-repeat genotype sequencing system 106 generates a probability of a nucleotide read originating from a candidate STR allele with a stutter model. In some implementations, the tandem-repeat genotype sequencing system 106 utilizes the following equation to generate the read probabilities:

$$P ( {r}_{k} \vert {a}_{i} , θ ) = ( 1 - u - d ) , {r}_{k} = {r}_{i}$$

where P(rₖ|aᵢ,θ) represents the probability of a nucleotide read having a number of nucleotide repeat units (rₖ) given a candidate STR allele i (aᵢ) and a stutter model (θ). In some implementations, the stutter model θ comprises the stutter model 616 illustrated in FIG. 6. As described previously, u represents the increased-repeat-unit probability, and d represents the decreased-repeat-unit probability. As shown, the tandem-repeat genotype sequencing system 106 utilizes the above equation when an STR length of a nucleotide read rₖ equals a candidate allele STR length rᵢ in STR candidate allele aᵢ.

In some implementations, the tandem-repeat genotype sequencing system 106 determines the STR length of a nucleotide read (rₖ) based on a motif length of the nucleotide repeat unit and the number of repeat units within the nucleotide read. Further, in the equation below and illustrated in FIG. 6 for the act 602, the motif length (m) comprises a value indicating a number of base pairs within a nucleotide repeat unit. For example, and as illustrated in FIG. 6, the STR length of the spanning read 608 equals fifteen, which is the product of the motif length (e.g., three) and the number of nucleotide repeat units (e.g., five). In another example, the tandem-repeat genotype sequencing system 106 determines that rₖ represents a number of nucleotide repeat units within a nucleotide read and rᵢ represents a number of nucleotide repeat units within a candidate STR allele. In these examples, and as illustrated in FIG. 6, rₖ of the spanning read 608 would simply equal five representing the number of nucleotide repeat units present within the spanning read 608.

As further illustrated in FIG. 6, the tandem-repeat genotype sequencing system 106 uses different formulations to generate the read probabilities when an STR length of a nucleotide read is greater than a candidate allele STR length (i.e., rₖ>rᵢ). For example, when the STR length of nucleotide read (rₖ) is greater than the candidate allele STR length (rᵢ), the tandem-repeat genotype sequencing system 106 utilizes the following equation to determine read probabilities:

$$u {q ( 1 - q )}^{( {r}_{k} - {r}_{i} ) / m - 1} , {r}_{k} > {r}_{i}$$

where u represents the increased-repeat-unit probability, q represents the size of stutter-induced changes, and m represents the motif length in base pairs.

Relatedly, the tandem-repeat genotype sequencing system 106 utilizes different formulations to generate the read probabilities when STR lengths of a nucleotide read is shorter than a candidate allele STR length (i.e., rₖ<rᵢ). For instance, when the STR length of a nucleotide read (rₖ) is less than the candidate allele STR length (rᵢ), the tandem-repeat genotype sequencing system 106 utilizes the following equation to determine read probabilities:

$$d {q ( 1 - q )}^{( {r}_{i} - {r}_{k} ) / m - 1} , {r}_{k} < {r}_{i}$$

where d represents the decreased-repeat-unit probability, q represents the size of stutter-induced changes, and m represents the motif length of the repeat unit in base pairs.

FIG. 6 further illustrates the tandem-repeat genotype sequencing system 106 performing the act 604 of determining the expected genotype probabilities utilizing the stutter model. In particular, the tandem-repeat genotype sequencing system 106 determines, utilizing the stutter model, the expected genotype probabilities based on the read probabilities. As shown, in some embodiments, the tandem-repeat genotype sequencing system 106 determines a probability of a candidate STR genotype 612 given a read distribution 614 and a stutter model θ. In some implementations, the tandem-repeat genotype sequencing system 106 utilizes the probabilities of reads generated as part of the act 602 to determine the read distribution 614. More specifically, the tandem-repeat genotype sequencing system 106 combines the read probabilities for nucleotide reads originating from all candidate STR alleles.

As illustrated in FIG. 6, in some embodiments, the tandem-repeat genotype sequencing system 106 determines expected genotype probabilities for the candidate STR genotypes. For instance, as shown in FIG. 6, the tandem-repeat genotype sequencing system 106 determines the expected probability of a candidate STR genotype Gᵢⱼ using the following equation:

$$P ( {G}_{i j} \vert R , θ ) = P ( {G}_{i j} ) {\prod }_{k = 1}^{n} {\sum }_{a ∈ i , j} P ( {r}_{k} \vert a , θ )$$

where θ represents the stutter model,

$${\prod }_{k = 1}^{n} {\sum }_{a ∈ i , j} P ( {r}_{k} \vert a , θ )$$

represents a probability of all reads originating from or belonging to the candidate STR genotype Gᵢⱼ comprising STR alleles (a) i and j, rₖ represents a number of nucleotide repeat units within a nucleotide read, n denotes the number of spanning nucleotide reads, and R represents the read distribution 614. Using a similar equation, the tandem-repeat genotype sequencing system 106 determines expected genotype probabilities for candidate STR genotypes Gᵢⱼ, Gⱼₖ, Gⱼⱼ, Gₖₖ, etc., depending on the candidate alleles exhibited by the spanning nucleotide reads for a genomic sample.

As previously mentioned, the tandem-repeat genotype sequencing system 106 performs a maximization stage of an EM algorithm. FIGS. 7A-7B illustrate the tandem-repeat genotype sequencing system 106 performing a maximization stage of an EM algorithm and updating the parameters of the stutter model in accordance with one or more embodiments of the present disclosure. By way of overview, FIGS. 7A-7B illustrate a series of acts comprising an act 702 of generating updated allele probabilities utilizing the stutter model and an act 704 of modifying the parameters of the stutter model.

As shown in FIG. 7A, the tandem-repeat genotype sequencing system 106 performs the act 702 of generating updated allele probabilities utilizing the stutter model. In particular, the tandem-repeat genotype sequencing system 106 generates, utilizing the stutter model, updated allele probabilities of candidate STR alleles among the spanning nucleotide reads based on the expected genotype probabilities. Previously, and as illustrated in FIG. 5, the tandem-repeat genotype sequencing system 106 determined initial allele probabilities of STR alleles and initial genotype probabilities based on observed numbers of nucleotide repeat units in spanning nucleotide reads. As part of performing the act 702 illustrated in FIG. 7A, the tandem-repeat genotype sequencing system 106 utilizes the expected genotype probabilities to generate updated allele probabilities utilizing the stutter model.

For example, the tandem-repeat genotype sequencing system 106 determines the updated allele probabilities by combining genotypes that have the candidate STR allele based on the expected genotype probabilities. In some embodiments, the tandem-repeat genotype sequencing system 106 generates the updated allele probabilities by utilizing the following equation:

$$P ( {a}_{i} ) = {\sum }_{j}^{A} P ( {G}_{i j} \vert R , θ )$$

where P(aᵢ) represents an updated allele probability for a candidate STR allele i, A represents a total number of candidate STR alleles, and P(Gᵢⱼ|R,θ) represents expected genotype probabilities given a read distribution R and the stutter model θ. More specifically, Gᵢⱼ denotes a candidate STR genotype with allele i and allele j, and R denotes a total number of spanning nucleotide reads. Using a similar equation, the tandem-repeat genotype sequencing system 106 determines updated allele probabilities for STR alleles aⱼ, aₖ, aₗ, etc., depending on the candidate alleles exhibited by the spanning nucleotide reads for a genomic sample.

As illustrated in FIG. 7B, the tandem-repeat genotype sequencing system 106 performs the act 704 of modifying the parameters of the stutter model. The tandem-repeat genotype sequencing system 106 modifies the parameters of the stutter model to maximize a total probability of the expected genotype probabilities based on the updated allele probabilities. The tandem-repeat genotype sequencing system 106 updates the following stutter model parameters: (u) an increased-repeat-unit probability, (d) a decreased-repeat-unit probability, and (q) a size of stutter-induced changes. The following paragraphs describe how the tandem-repeat genotype sequencing system 106 modifies each of these stutter model parameters in accordance with one or more implementations.

As shown in FIG. 7B, the tandem-repeat genotype sequencing system 106 adjusts the increased-repeat-unit probability (u) based on updated allele probabilities of candidate STR alleles among the spanning nucleotide reads and a first subset of spanning nucleotide reads comprising more nucleotide repeat units than the candidate STR alleles. To illustrate, the tandem-repeat genotype sequencing system 106 identifies a first set of spanning nucleotide reads 708 that comprise more nucleotide repeat units than the candidate STR alleles. For example, as illustrated in FIG. 7B, spanning nucleotide reads in the first set of spanning nucleotide reads 708 have more nucleotide repeat units than the candidate STR allele i. In some implementations, the tandem-repeat genotype sequencing system 106 utilizes the following formulation to modify the increased-repeat-unit probability (u):

$$u = {\sum }_{i}^{A} {\sum }_{j}^{A} P ( {G}_{i j} \vert R , θ ) {\sum }_{k = 1}^{n} {\sum }_{a ∈ i , j} P ( {r}_{k} \vert a , θ ) \vert ( {r}_{k} > {r}_{i} )$$

$$where {\sum }_{i}^{A} {\sum }_{j}^{A} P ( {G}_{i j} \vert R , θ )$$

represents a computation of a probabilities of all candidate STR genotypes

$${G}_{i j} , and {\sum }_{k = 1}^{n} {\sum }_{a ∈ i , j} P ( {r}_{k} \vert a , θ ) \vert ( {r}_{k} > {r}_{i} )$$

represents the stutter model of all spanning nucleotide reads with more nucleotide repeat units than a given candidate STR genotype Gᵢⱼ. Notably, in adjusting the increased-repeat-unit probability (u), the tandem-repeat genotype sequencing system 106 analyzes only the first set of spanning nucleotide reads that have more repeat units than a given candidate STR allele.

Similarly, the tandem-repeat genotype sequencing system 106 modifies the decreased-repeat-unit probability (d) based on updated allele probabilities and a second subset of spanning nucleotide reads comprising fewer nucleotide repeat units than the candidate STR alleles. For example, and as illustrated in FIG. 7B, the tandem-repeat genotype sequencing system 106 identifies a second subset of spanning nucleotide reads 706 that contain fewer nucleotide repeat units than the candidate STR allele i. In some implementations, the tandem-repeat genotype sequencing system 106 utilizes the following formation to modify the decreased-repeat-unit probability (d):

$$d = {\sum }_{i}^{A} {\sum }_{j}^{A} P ( {G}_{i j} \vert R , θ ) {\sum }_{k = 1}^{n} {\sum }_{a ∈ i , j} P ( {r}_{k} \vert a , θ ) \vert ( {r}_{k} < {r}_{i} )$$

$$where {\sum }_{i}^{A} {\sum }_{j}^{A} P ( {G}_{i j} \vert R , θ )$$

represents a computation of a probabilities of all candidate STR genotypes

$${G}_{i j} , and {\sum }_{k = 1}^{n} {\sum }_{a ∈ i , j} P ( {r}_{k} \vert a , θ ) \vert ( {r}_{k} > {r}_{i} )$$

represents the stutter model of all spanning nucleotide reads with more nucleotide repeat units than a given candidate STR genotype Gᵢⱼ. When adjusting the decreased-repeat-unit probability (d), the tandem-repeat genotype sequencing system 106 analyzes only the second set of spanning nucleotide reads that have fewer repeat units than the given candidate STR allele.

As further shown in FIG. 7B, the tandem-repeat genotype sequencing system 106 modifies the size of the stutter-induced changes (q). In particular, the tandem-repeat genotype sequencing system 106 adjusts the size of the stutter-induced changes based on an inverse of a mean weighted step size for nucleotide reads exhibiting stutter-induced changes to nucleotide repeat units. Generally, the tandem-repeat genotype sequencing system 106 analyzes all the spanning reads as part of adjusting q. The tandem-repeat genotype sequencing system 106 considers all spanning nucleotide reads that have a different number of repeat units than the candidate STR allele (rₖ≠rᵢ). The tandem-repeat genotype sequencing system 106 also determines a magnitude of the difference between nucleotide reads in the spanning nucleotide reads and the candidate STR allele (rₖ−rᵢ). For example, and as shown in FIG. 7B, the tandem-repeat genotype sequencing system 106 identifies spanning nucleotide reads 710 that contain more or fewer nucleotide repeat units than the candidate STR allele i. In some implementations, the tandem-repeat genotype sequencing system 106 utilizes the following formulation to modify the size of the stutter-induced changes (q):

$$q = \frac{{\sum }_{i}^{A} {\sum }_{j}^{A} P ( {G}_{ij} \vert R , θ ) {\sum }_{k = 1}^{n} {\sum }_{a ∈ i , j} P ( {r}_{k} \vert a , θ ) \vert ( {r}_{k} \neq {r}_{i} )}{{\sum }_{i}^{A} {\sum }_{j}^{A} P ( {G}_{ij} \vert R , θ ) {\sum }_{k = 1}^{n} {\sum }_{a ∈ i , j} P ( {r}_{k} \vert a , θ ) \vert {r}_{k} - {r}_{i} \vert}$$

$$where {\sum }_{i}^{A} {\sum }_{j}^{A} P ( {G}_{i j} \vert R , θ )$$

represents a computation of a probabilities of all candidate STR genotypes

$${G}_{i j} , {\sum }_{k = 1}^{n} {\sum }_{a ∈ i , j} P ( {r}_{k} \vert a , θ ) \vert ( {r}_{k} \neq {r}_{i} )$$

represents the stutter model of spanning nucleotide reads with different numbers (either more or fewer) of nucleotide repeat units than a given candidate STR genotype

$${G}_{i j} , and {\sum }_{k = 1}^{n} {\sum }_{a ∈ i , j} P ( {r}_{k} \vert a , θ ) \vert {r}_{k} \neq {r}_{i} \vert$$

represents a magnitude of differences between the number of nucleotide repeat units within the spanning nucleotide reads. Using a similar equations for d, u, and q, the tandem-repeat genotype sequencing system 106 likewise determines updated parameters d, u, and q for candidate STR genotypes Gᵢᵢ, Gⱼₖ, Gⱼⱼ, Gₖₖ, etc., depending on the candidate alleles exhibited by the spanning nucleotide reads for a genomic sample.

As previously described, the tandem-repeat genotype sequencing system 106 determines a genotype call from the candidate STR genotypes that the genomic sample comprises one or more alleles at the STR region based on the converged genotype probabilities. FIG. 8 illustrates the tandem-repeat genotype sequencing system 106 determining the genotype call from candidate STR genotypes in accordance with one or more embodiments of the present disclosure. By way of overview, FIG. 8 illustrates a series of acts including an act 802 of determining that the genotype probabilities of STR genotypes have converged and an act 804 of selecting a candidate STR genotype with the maximum likelihood.

As previously described, the tandem-repeat genotype sequencing system 106 iteratively performs the expectation and maximization stages of the described EM algorithm until reaching converged genotype probabilities of STR genotypes. For example, the tandem-repeat genotype sequencing system 106 iteratively performs the acts illustrated in FIG. 6 and FIGS. 7A-7B to generate expected genotype probabilities and modify parameters of the stutter model. The tandem-repeat genotype sequencing system 106 evaluates the expected genotype probabilities generated utilizing the modified stutter model.

As shown in FIG. 8, the tandem-repeat genotype sequencing system 106 performs the act 802 of determining that the genotype probabilities of STR genotypes have converged. In some implementations, the tandem-repeat genotype sequencing system 106 determines that the genotype probabilities of candidate STR genotypes have converged based on determining that a product of the expected genotype probabilities in successive iterations fall within a threshold convergence range. As illustrated in FIG. 8, the tandem-repeat genotype sequencing system 106 may utilize the following equation to determine a product of expected genotype probabilities for candidate STR genotypes in single iteration:

$${\prod }_{{G}_{i , j}} P ( {G}_{i j} \vert R , θ )$$

Generally, the tandem-repeat genotype sequencing system 106 generates expected genotype probabilities for all possible candidate STR genotypes given a read distribution R and stutter model θ. The tandem-repeat genotype sequencing system 106 determines a product of all the expected genotype probabilities within an iteration.

In some implementations, the tandem-repeat genotype sequencing system 106 determines a difference between the products of expected genotype probabilities within successive iterations. The tandem-repeat genotype sequencing system 106 determines whether the difference between the products of expected genotype probabilities within successive iterations falls within a threshold convergence range. Based on determining that products of the expected genotype probabilities in successive iterations fall outside a threshold convergence range, the tandem-repeat genotype sequencing system 106 performs another iteration of the EM algorithm.

In some examples, the tandem-repeat genotype sequencing system 106 determines the threshold convergence range based on user input. For example, the tandem-repeat genotype sequencing system 106 can receive, from a client device (e.g., the client device 114) associated with a user, a desired threshold convergence range. In other implementations, the tandem-repeat genotype sequencing system 106 determines the threshold convergence range without user input. By contrast, in some embodiments, the tandem-repeat genotype sequencing system 106 continues to perform expectation and maximization stages for a set or pre-determined number of iterations and, afterwards, selects the expected genotype probabilities from the last iterations upon which to determine a genotype call. Such a set or pre-determined number of iterations can be selected by a client device.

In some embodiments, based on determining that the products of the expected genotype probabilities in successive iterations fall within a threshold convergence range, the tandem-repeat genotype sequencing system 106 proceeds to the act 804 illustrated in FIG. 8. In particular, the tandem-repeat genotype sequencing system 106 performs the act 804 of selecting a candidate STR genotype with the maximum likelihood. More specifically, the tandem-repeat genotype sequencing system 106 selects a most likely candidate STR genotype from the candidate STR genotypes generated within an iteration having the highest expected genotype probability. The tandem-repeat genotype sequencing system 106 determines the genotype call based on the most likely candidate STR genotype. As illustrated in FIG. 8, the tandem-repeat genotype sequencing system 106 determines the most likely candidate STR genotype comprises a first allele having four repeat units and a second allele having eight repeat units. The tandem-repeat genotype sequencing system 106 generates a genotype call reflecting the sequence of the most likely candidate STR genotype.

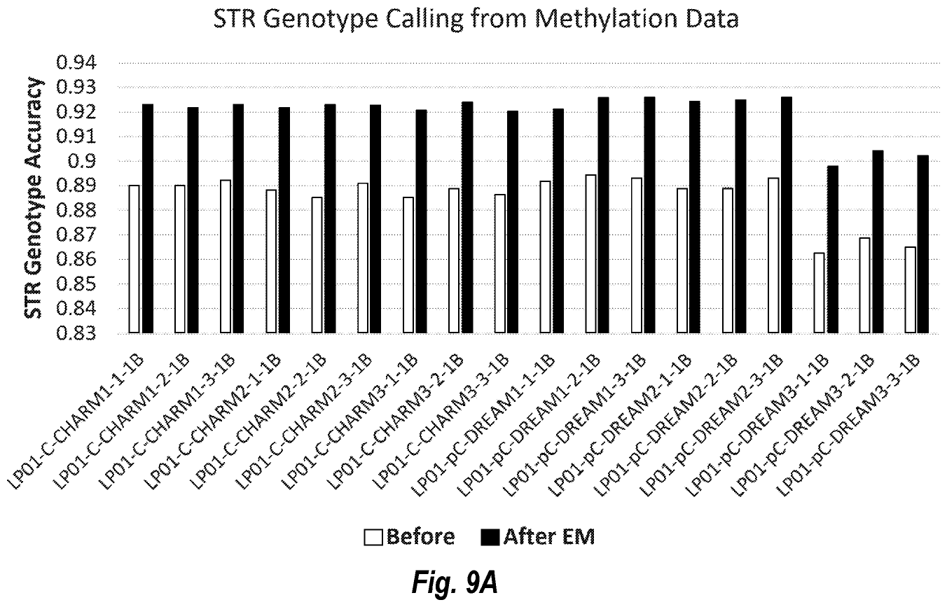

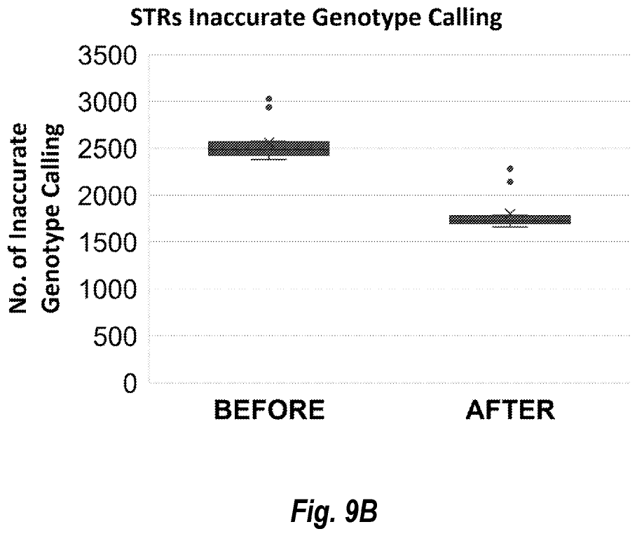

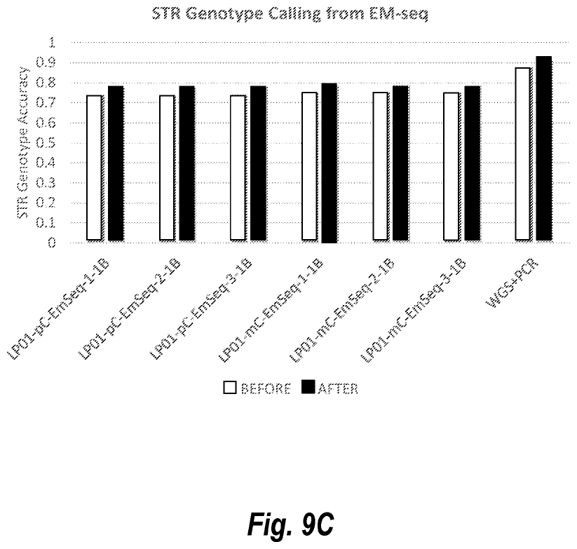

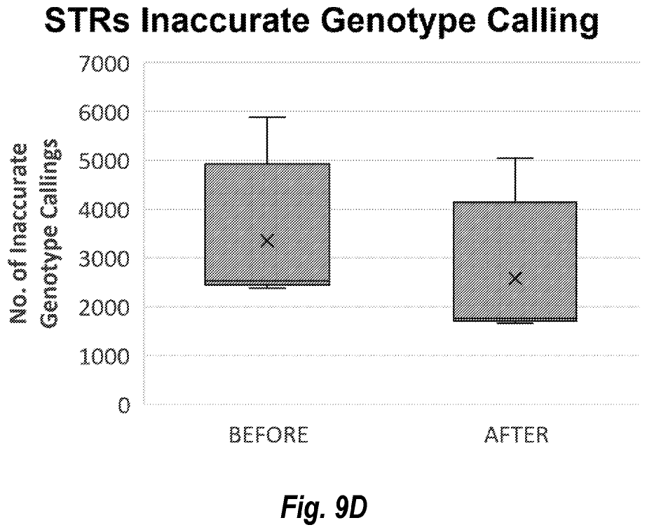

As mentioned, previously, the tandem-repeat genotype sequencing system 106 makes improvements to the accuracy of STR genotyping. FIGS. 9A-9D illustrate a series of graphs indicating the tandem-repeat genotype sequencing system 106 more accurately genotyping STRs from methylation data relative to existing sequencing systems in accordance with one or more embodiments of the present disclosure. FIGS. 9A-9B illustrate the tandem-repeat genotype sequencing system 106 improving accuracy and decreasing numbers of inaccurate genotype calls from methylation data comprising methylated cytosines in accordance with one or more embodiments. FIGS. 9C-9D illustrate the tandem-repeat genotype sequencing system 106 improving accuracy and decreasing numbers of inaccurate genotype calls from methylation data comprising unmethylated cytosines in accordance with one or more embodiments.

FIG. 9A illustrates the tandem-repeat genotype sequencing system 106 improving STR genotyping accuracy from methylation data from methylation assays that modify or convert methylated cytosines during library prep. For example, the methylation assays depicted in FIG. 9A include Comprehensive High-Throughput Arrays for Relative Methylation (CHARM) assays and Digital Restriction Enzyme Analysis of Methylation (DREAM) assays. In the sample name LP01-C-CHARM1-2-1B, LP01 represents genome NA12878, C-CHARM1 represents a CHARM assay, and 2 indicates the 2nd replication of the assay. As shown, by utilizing the EM algorithm, the tandem-repeat genotype sequencing system 106 improves STR genotype calling accuracy by approximately 3%. More specifically, FIG. 9A illustrates a difference in STR genotyping accuracy before utilization of the EM algorithm and after utilization of the EM algorithm. As further illustrated, the tandem-repeat genotype sequencing system 106 can achieve STR genotyping accuracies up to 92%.

As mentioned, the tandem-repeat genotype sequencing system 106 also decreases the number of inaccurate STR genotype calling from nucleotide reads sequenced as part of a methylation sequencing assay. FIG. 9B illustrates differences in the number of inaccurate STR genotype calling before utilization of an EM algorithm and after utilization of the EM algorithm. As illustrated, the tandem-repeat genotype sequencing system 106 decreases the number of inaccurate STR genotype calling by approximately 30%, that is from approximately 2,500 inaccurate genotype calls to approximately 1,750 inaccurate genotype calls.

The tandem-repeat genotype sequencing system 106 also improves the accuracy of STR genotype calling for methylation data from methylation sequencing assays that modify or convert unmethylated cytosines. FIG. 9C illustrates the tandem-repeat genotype sequencing system 106 improving STR genotype calling accuracies from C to T methylation sequencing assays. FIG. 9C portrays differences in STR genotyping accuracy for various methylation assays before and after utilization of the EM algorithm. As indicated by the graph, researcher performed Enzymatic Methyl-seq (EM-seq) as a methylation sequencing assay. For instance, the researchers performed EM-seq as described by Romualdas Vaisvila et al., Enzymatic Methyl Sequencing Detects DNA Methylation at Single-Base Resolution from Picograms of DNA, 30 Genome Research 1280-1289 (2021), which is hereby incorporated by reference in its entirety.

As shown in FIG. 9C, the tandem-repeat genotype sequencing system 106 improves STR genotype calling accuracy by approximately 3% in EM-seq assay. FIG. 9C further illustrates improvements to STR genotyping accuracy for whole genome sequencing (WGS) with a PCR step.

In addition to measuring accuracy in terms of percentage or probability for EM-seq assays in FIG. 9C, researchers measured accuracy for EM-seq in terms of number of inaccurate genotype calls in FIG. 9D. FIG. 9D illustrates the tandem-repeat genotype sequencing system 106 decreasing a number of inaccurate STR genotype callings from methylation data that converts unmethylated cytosines. For example, FIG. 9D shows a decrease in number of inaccurate STR genotype calls after application of the EM algorithm disclosed herein.

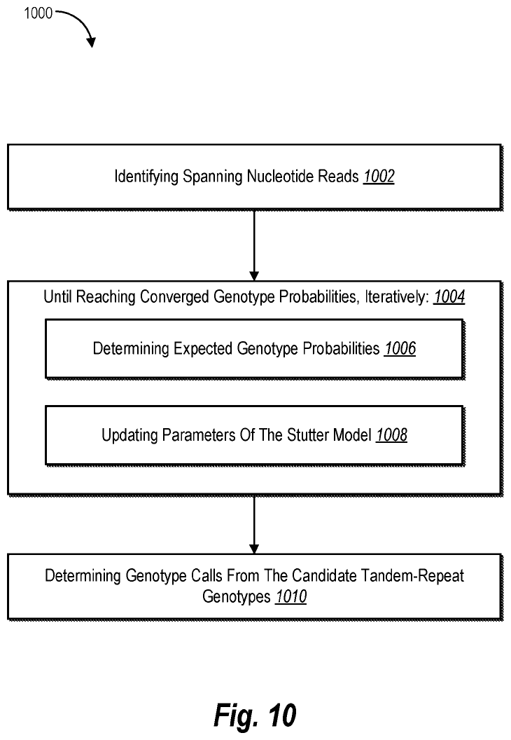

FIGS. 1-9D, the corresponding text, and the examples provide a number of different methods, systems, devices, and non-transitory computer-readable media of the tandem-repeat genotype sequencing system 106. In addition to the foregoing, one or more implementations can also be described in terms of flowcharts comprising acts for accomplishing a particular result, as shown in FIG. 10. FIG. 10 illustrates a flowchart of a series of acts 1000 of determining genotype calls from candidate STR genotypes in accordance with one or more embodiments of the present disclosure. While FIG. 10 illustrates acts according to one embodiment, alternative embodiments may omit, add to, reorder, and/or modify any of the acts shown in FIG. 10. The acts of FIG. 10 can be performed as part of a method. Alternatively, a non-transitory computer readable storage medium can comprise instructions that, when executed by one or more processors, cause a computing device or a system to perform the acts depicted in FIG. 10. In still further embodiments, a system comprising at least one processor and a non-transitory computer readable medium comprising instructions that, when executed by one or more processors, cause the system to perform the acts of FIG. 10.

As shown in FIG. 10, the series of acts 1000 includes an act 1002 of identifying spanning nucleotide reads and an act 1004 of until reaching converged genotype probabilities, iteratively performing an act 1006 of determining expected genotype probabilities and an act 1008 of updating parameters of the stutter model. The series of acts 1000 also includes an act 1010 of determining genotype calls from the candidate tandem-repeat genotypes. For example, the series of acts 1000 can include acts to perform any of the operations described in the following clauses:

CLAUSE 1. A method comprising:
identifying, from nucleotide reads sequenced for a genomic sample, spanning nucleotide reads that cover a tandem-repeat region;
until reaching converged genotype probabilities of tandem-repeat genotypes, iteratively:
   determining, for the genomic sample and utilizing a stutter model, expected genotype probabilities of candidate tandem-repeat genotypes based on differing numbers of nucleotide repeat units in the spanning nucleotide reads; and
   updating parameters of the stutter model based on the expected genotype probabilities; and
      determining a genotype call from the candidate tandem-repeat genotypes that the genomic sample comprises one or more tandem-repeat alleles at the tandem-repeat region based on the converged genotype probabilities.

CLAUSE 2. The method of clause 1, wherein:
the tandem-repeat region comprises a short tandem repeat (STR) or microsatellite region, a minisatellite region, a variable number tandem repeat (VNTR) region, or a guanine quadruplex region;
the one or more tandem-repeat genotypes comprise STR or microsatellite genotypes, minisatellite genotypes, VNTR genotypes, or guanine-quadruplex genotypes; and
   the tandem-repeat alleles comprise STR or microsatellite alleles, minisatellite alleles, VNTR alleles, or guanine-quadruplex alleles.

CLAUSE 3. The method of clause 1, wherein identifying the spanning nucleotide reads comprises extracting the spanning nucleotide reads from the nucleotide reads sequenced in a methylation assay for the genomic sample.

CLAUSE 4. The method of clause 1, further comprising determining the candidate tandem-repeat genotypes by:
determining a set of candidate tandem-repeat alleles based on the differing numbers of nucleotide repeat units in the spanning nucleotide reads; and
generating, from the set of candidate tandem-repeat alleles, combinations of two candidate tandem-repeat alleles as part of the candidate tandem-repeat genotypes.

CLAUSE 5. The method of clause 1, wherein the parameters of the stutter model comprise:
an increased-repeat-unit probability of a given nucleotide read comprising more nucleotide repeat units than a reference genome within a corresponding tandem-repeat region;
a decreased-repeat-unit probability of the given nucleotide read comprising fewer nucleotide repeat units than the reference genome within the corresponding tandem-repeat region; and
a size of stutter-induced changes in the spanning nucleotide reads.

CLAUSE 6. The method of clause 5, wherein updating the parameters of the stutter model comprises:
adjusting the increased-repeat-unit probability based on updated allele probabilities of candidate tandem-repeat alleles among the spanning nucleotide reads and a first subset of spanning nucleotide reads comprising more nucleotide repeat units than the candidate tandem-repeat alleles;
adjusting the decreased-repeat-unit probability based on the updated allele probabilities and a second subset of spanning nucleotide reads comprising fewer nucleotide repeat units than the candidate tandem-repeat alleles; and
adjusting the size of the stutter-induced changes based on an inverse of a mean weighted step size for nucleotide reads exhibiting stutter-induced changes to nucleotide repeat units.

CLAUSE 7. The method of clause 1, further comprising:
initializing, for the stutter model, a value for an increased-repeat-unit probability of a given nucleotide read comprising more nucleotide repeat units than a reference genome within a corresponding tandem-repeat region; and
initializing, for the stutter model, a value for a decreased-repeat-unit probability of the given nucleotide read comprising fewer nucleotide repeat units than the reference genome within the corresponding tandem-repeat region, wherein the initialized value for the decreased-repeat-unit probability exceeds the initialized value for the increased-repeat-unit probability.

CLAUSE 8. The method of clause 1, further comprising:
determining initial allele probabilities of tandem-repeat alleles for a set of observed tandem-repeat alleles in the spanning nucleotide reads; and
determining initial genotype probabilities based on the initial allele probabilities.

CLAUSE 9. The method of clause 1, wherein determining the expected genotype probabilities of tandem-repeat genotypes comprises performing an expectation stage of an expectation-maximization (EM) algorithm by:
generating, utilizing the stutter model, read probabilities of nucleotide reads originating from a set of candidate tandem-repeat alleles; and
determining, utilizing the stutter model, the expected genotype probabilities based on the read probabilities.

CLAUSE 10. The method of clause 9, wherein updating the parameters of the stutter model further comprises performing a maximization stage of an EM algorithm by:
generating, utilizing the stutter model, updated allele probabilities of candidate tandem-repeat alleles among the spanning nucleotide reads based on the expected genotype probabilities; and
modifying the parameters of the stutter model to maximize a total probability of the expected genotype probabilities based on the updated allele probabilities.

CLAUSE 11. The method of clause 1, further comprising determining that the expected genotype probabilities of candidate tandem-repeat genotypes have converged based on determining that products of the expected genotype probabilities in successive iterations fall within a threshold convergence range.

CLAUSE 12. A method comprising:
identifying, from nucleotide reads sequenced for a genomic sample, spanning nucleotide reads that cover a short tandem repeat (STR) region;
until reaching converged genotype probabilities of STR genotypes, iteratively:
   determining, for the genomic sample and utilizing a stutter model, expected genotype probabilities of candidate STR genotypes based on differing numbers of nucleotide repeat units in the spanning nucleotide reads; and
   updating parameters of the stutter model based on the expected genotype probabilities; and
determining a genotype call from the candidate STR genotypes that the genomic sample comprises one or more STR alleles at the STR region based on the converged genotype probabilities.

CLAUSE 13. The method of clause 12, further comprising determining the candidate STR genotypes by:
determining a set of candidate STR alleles based on the differing numbers of nucleotide repeat units in the spanning nucleotide reads; and
generating, from the set of candidate STR alleles, combinations of two candidate STR alleles as part of the candidate STR genotypes.

CLAUSE 14. The method of clause 12, wherein the parameters of the stutter model comprise:
an increased-repeat-unit probability of a given nucleotide read comprising more nucleotide repeat units than a reference genome within a corresponding STR region;
a decreased-repeat-unit probability of the given nucleotide read comprising fewer nucleotide repeat units than the reference genome within the corresponding STR region; and
a size of stutter-induced changes in the spanning nucleotide reads.

CLAUSE 15. The method of clause 14, further comprising updating the parameters of the stutter model by:
adjusting the increased-repeat-unit probability based on updated allele probabilities of candidate STR alleles among the spanning nucleotide reads and a first subset of spanning nucleotide reads comprising more nucleotide repeat units than the candidate STR alleles;
adjusting the decreased-repeat-unit probability based on the updated allele probabilities and a second subset of spanning nucleotide reads comprising fewer nucleotide repeat units than the candidate STR alleles; and
adjusting the size of the stutter-induced changes based on an inverse of a mean weighted step size for nucleotide reads exhibiting stutter-induced changes to nucleotide repeat units.

CLAUSE 16. The method of clause 12, further comprising:
initializing, for the stutter model, a value for an increased-repeat-unit probability of a given nucleotide read comprising more nucleotide repeat units than a reference genome within a corresponding STR region; and
initializing, for the stutter model, a value for a decreased-repeat-unit probability of the given nucleotide read comprising fewer nucleotide repeat units than the reference genome within the corresponding STR region, wherein the initialized value for the decreased-repeat-unit probability exceeds the initialized value for the increased-repeat-unit probability.

CLAUSE 17. The method of clause 12, further comprising:
determining initial allele probabilities of STR alleles for a set of observed STR alleles in the spanning nucleotide reads; and
determining initial genotype probabilities based on the initial allele probabilities.

CLAUSE 18. The method of clause 12, further comprising determining the expected genotype probabilities of STR genotypes by performing an expectation stage of an expectation-maximization (EM) algorithm comprising:
generating, utilizing the stutter model, read probabilities of nucleotide reads originating from a set of candidate STR alleles; and
determining, utilizing the stutter model, the expected genotype probabilities based on the read probabilities.

CLAUSE 19. The method of clause 18, further comprising updating the parameters of the stutter model further by performing a maximization stage of an EM algorithm comprising:
generating, utilizing the stutter model, updated allele probabilities of candidate STR alleles among the spanning nucleotide reads based on the expected genotype probabilities; and
modifying the parameters of the stutter model to maximize a total probability of the expected genotype probabilities based on the updated allele probabilities.

CLAUSE 20. The method of clause 18, further comprising determining that the expected genotype probabilities of candidate STR genotypes have converged based on determining that products of the expected genotype probabilities in successive iterations fall within a threshold convergence range.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. No. 7,329,492 and U.S. Pat. No. 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the tandem-repeat genotype sequencing system 106 can include software, hardware, or both. For example, the components of the tandem-repeat genotype sequencing system 106 can include one or more instructions stored on a computer-readable storage medium and executable by processors of one or more computing devices (e.g., the client device 114). When executed by the one or more processors, the computer-executable instructions of the tandem-repeat genotype sequencing system 106 can cause the computing devices to perform the bubble detection methods described herein. Alternatively, the components of the tandem-repeat genotype sequencing system 106 can comprise hardware, such as special purpose processing devices to perform a certain function or group of functions. Additionally, or alternatively, the components of the tandem-repeat genotype sequencing system 106 can include a combination of computer-executable instructions and hardware.

Furthermore, the components of the tandem-repeat genotype sequencing system 106 performing the functions described herein with respect to the tandem-repeat genotype sequencing system 106 may, for example, be implemented as part of a stand-alone application, as a module of an application, as a plug-in for applications, as a library function or functions that may be called by other applications, and/or as a cloud-computing model. Thus, components of the tandem-repeat genotype sequencing system 106 may be implemented as part of a stand-alone application on a personal computing device or a mobile device. Additionally, or alternatively, the components of the tandem-repeat genotype sequencing system 106 may be implemented in any application that provides sequencing services including, but not limited to Illumina BaseSpace, Illumina DRAGEN, Illumina NextSeq, Illumina TruSeq, or Illumina TruSight software. “Illumina,” “BaseSpace,” “DRAGEN,” “NextSeq,” “TruSeq,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 11 illustrates a block diagram of a computing device 1100 that may be configured to perform one or more of the processes described above. One will appreciate that one or more computing devices such as the computing device 1100 may implement the tandem-repeat genotype sequencing system 106. As shown by FIG. 11, the computing device 1100 can comprise a processor 1102, a memory 1104, a storage device 1106, an I/O interface 1108, and a communication interface 1110, which may be communicatively coupled by way of a communication infrastructure 1112. In certain embodiments, the computing device 1100 can include fewer or more components than those shown in FIG. 11. The following paragraphs describe components of the computing device 1100 shown in FIG. 11 in additional detail.

In one or more embodiments, the processor 1102 includes hardware for executing instructions, such as those making up a computer program. As an example, and not by way of limitation, to execute instructions for dynamically modifying workflows, the processor 1102 may retrieve (or fetch) the instructions from an internal register, an internal cache, the memory 1104, or the storage device 1106 and decode and execute them. The memory 1104 may be a volatile or non-volatile memory used for storing data, metadata, and programs for execution by the processor(s). The storage device 1106 includes storage, such as a hard disk, flash disk drive, or other digital storage device, for storing data or instructions for performing the methods described herein.

The I/O interface 1108 allows a user to provide input to, receive output from, and otherwise transfer data to and receive data from computing device 1100. The I/O interface 1108 may include a mouse, a keypad or a keyboard, a touch screen, a camera, an optical scanner, network interface, modem, other known I/O devices or a combination of such I/O interfaces. The I/O interface 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1108 is configured to provide graphical data to a display for presentation to a user. The graphical data may be representative of one or more graphical user interfaces and/or any other graphical content as may serve a particular implementation.

The communication interface 1110 can include hardware, software, or both. In any event, the communication interface 1110 can provide one or more interfaces for communication (such as, for example, packet-based communication) between the computing device 1100 and one or more other computing devices or networks. As an example, and not by way of limitation, the communication interface 1110 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1110 may facilitate communications with various types of wired or wireless networks. The communication interface 1110 may also facilitate communications using various communication protocols. The communication infrastructure 1112 may also include hardware, software, or both that couples components of the computing device 1100 to each other. For example, the communication interface 1110 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

1-20. (canceled)

21. A system comprising:
at least one processor; and
a non-transitory computer readable medium comprising instructions that, when executed by the at least one processor, cause the system to:
identify, from nucleotide reads sequenced for a genomic sample, spanning nucleotide reads that cover a tandem-repeat region;
until reaching converged genotype probabilities of tandem-repeat genotypes, iteratively:
determine, for the genomic sample and utilizing a stutter model, expected genotype probabilities of candidate tandem-repeat genotypes based on differing numbers of nucleotide repeat units in the spanning nucleotide reads; and
update parameters of the stutter model based on the expected genotype probabilities; and
determine a genotype call from the candidate tandem-repeat genotypes that the genomic sample comprises one or more tandem-repeat alleles at the tandem-repeat region based on the converged genotype probabilities.

22. The system of claim 21, wherein:
the tandem-repeat region comprises a short tandem repeat (STR) or microsatellite region, a minisatellite region, a variable number tandem repeat (VNTR) region, or a guanine quadruplex region;
the tandem-repeat genotypes comprise STR or microsatellite genotypes, minisatellite genotypes, VNTR genotypes, or guanine-quadruplex genotypes; and
the one or more tandem-repeat alleles comprise STR or microsatellite alleles, minisatellite alleles, VNTR alleles, or guanine-quadruplex alleles.

23. The system of claim 21, further comprising further comprising instructions that, when executed by the at least one processor, cause the system to determine the candidate tandem-repeat genotypes by:
determining a set of candidate tandem-repeat alleles based on the differing numbers of nucleotide repeat units in the spanning nucleotide reads; and
generating, from the set of candidate tandem-repeat alleles, combinations of two candidate tandem-repeat alleles as part of the candidate tandem-repeat genotypes.

24. The system of claim 21, wherein the parameters of the stutter model comprise:
an increased-repeat-unit probability of a given nucleotide read comprising more nucleotide repeat units than a reference genome within a corresponding tandem-repeat region;
a decreased-repeat-unit probability of the given nucleotide read comprising fewer nucleotide repeat units than the reference genome within the corresponding tandem-repeat region; and
a size of stutter-induced changes in the spanning nucleotide reads.

25. The system of claim 24, further comprising instructions that, when executed by the at least one processor, cause the system to update the parameters of the stutter model by:
adjusting the increased-repeat-unit probability based on updated allele probabilities of candidate tandem-repeat alleles among the spanning nucleotide reads and a first subset of spanning nucleotide reads comprising more nucleotide repeat units than the candidate tandem-repeat alleles;
adjusting the decreased-repeat-unit probability based on the updated allele probabilities and a second subset of spanning nucleotide reads comprising fewer nucleotide repeat units than the candidate tandem-repeat alleles; and
adjusting the size of the stutter-induced changes based on an inverse of a mean weighted step size for nucleotide reads exhibiting stutter-induced changes to nucleotide repeat units.

26. The system of claim 21, further comprising instructions that, when executed by the at least one processor, cause the system to:
initialize, for the stutter model, a value for an increased-repeat-unit probability of a given nucleotide read comprising more nucleotide repeat units than a reference genome within a corresponding tandem-repeat region; and
initialize, for the stutter model, a value for a decreased-repeat-unit probability of the given nucleotide read comprising fewer nucleotide repeat units than the reference genome within the corresponding tandem-repeat region, wherein the initialized value for the decreased-repeat-unit probability exceeds the initialized value for the increased-repeat-unit probability.

27. The system of claim 21, further comprising instructions that, when executed by the at least one processor, cause the system to:
determine initial allele probabilities of tandem-repeat alleles for a set of observed tandem-repeat alleles in the spanning nucleotide reads; and
determine initial genotype probabilities based on the initial allele probabilities.

28. The system of claim 21, further comprising instructions that, when executed by the at least one processor, cause the system to determine the expected genotype probabilities of tandem-repeat genotypes by performing an expectation stage of an expectation-maximization (EM) algorithm comprising:
generating, utilizing the stutter model, read probabilities of nucleotide reads originating from a set of candidate tandem-repeat alleles; and
determining, utilizing the stutter model, the expected genotype probabilities based on the read probabilities.

29. The system of claim 27, further comprising instructions that, when executed by the at least one processor, cause the system to update the parameters of the stutter model further by performing a maximization stage of an EM algorithm comprising:
generating, utilizing the stutter model, updated allele probabilities of candidate tandem-repeat alleles among the spanning nucleotide reads based on the expected genotype probabilities; and
modifying the parameters of the stutter model to maximize a total probability of the expected genotype probabilities based on the updated allele probabilities.

30. The system of claim 21, further comprising instructions that, when executed by the at least one processor, cause the system to determine that the expected genotype probabilities of candidate tandem-repeat genotypes have converged based on determining that products of the expected genotype probabilities in successive iterations fall within a threshold convergence range.

31. The system of claim 21, further comprising instructions that, when executed by the at least one processor, cause the system to identify the spanning nucleotide reads by extracting the spanning nucleotide reads from the nucleotide reads sequenced in a methylation assay for the genomic sample.

32. The system of claim 21, further comprising instructions that, when executed by the at least one processor, cause the system to determine that the expected genotype probabilities of candidate tandem-repeat genotypes have converged based on determining that products of the expected genotype probabilities in successive iterations fall within a threshold convergence range.

33. A non-transitory computer-readable medium comprising instructions that, when executed by at least one processor, cause a computing device to:
identify, from nucleotide reads sequenced for a genomic sample, spanning nucleotide reads that cover a tandem-repeat region;
until reaching converged genotype probabilities of tandem-repeat genotypes, iteratively:
determine, for the genomic sample and utilizing a stutter model, expected genotype probabilities of candidate tandem-repeat genotypes based on differing numbers of nucleotide repeat units in the spanning nucleotide reads; and
update parameters of the stutter model based on the expected genotype probabilities; and
determine a genotype call from the candidate tandem-repeat genotypes that the genomic sample comprises one or more tandem-repeat alleles at the tandem-repeat region based on the converged genotype probabilities.

34. The non-transitory computer-readable medium of claim 33, wherein:
the tandem-repeat region comprises a short tandem repeat (STR) or microsatellite region, a minisatellite region, a variable number tandem repeat (VNTR) region, or a guanine quadruplex region;
the tandem-repeat genotypes comprise STR or microsatellite genotypes, minisatellite genotypes, VNTR genotypes, or guanine-quadruplex genotypes; and
the one or more tandem-repeat alleles comprise STR or microsatellite alleles, minisatellite alleles, VNTR alleles, or guanine-quadruplex alleles.

35. The non-transitory computer-readable medium of claim 33, further comprising further comprising instructions that, when executed by the at least one processor, cause the computing device to determine the candidate tandem-repeat genotypes by:
determining a set of candidate tandem-repeat alleles based on the differing numbers of nucleotide repeat units in the spanning nucleotide reads; and
generating, from the set of candidate tandem-repeat alleles, combinations of two candidate tandem-repeat alleles as part of the candidate tandem-repeat genotypes.

36. The non-transitory computer-readable medium of claim 33, wherein the parameters of the stutter model comprise:
an increased-repeat-unit probability of a given nucleotide read comprising more nucleotide repeat units than a reference genome within a corresponding tandem-repeat region;
a decreased-repeat-unit probability of the given nucleotide read comprising fewer nucleotide repeat units than the reference genome within the corresponding tandem-repeat region; and
a size of stutter-induced changes in the spanning nucleotide reads.

37. The non-transitory computer-readable medium of claim 36, further comprising instructions that, when executed by the at least one processor, cause the computing device to update the parameters of the stutter model by:
adjusting the increased-repeat-unit probability based on updated allele probabilities of candidate tandem-repeat alleles among the spanning nucleotide reads and a first subset of spanning nucleotide reads comprising more nucleotide repeat units than the candidate tandem-repeat alleles;
adjusting the decreased-repeat-unit probability based on the updated allele probabilities and a second subset of spanning nucleotide reads comprising fewer nucleotide repeat units than the candidate tandem-repeat alleles; and
adjusting the size of the stutter-induced changes based on an inverse of a mean weighted step size for nucleotide reads exhibiting stutter-induced changes to nucleotide repeat units.

38. A method comprising:
identifying, from nucleotide reads sequenced for a genomic sample, spanning nucleotide reads that cover a tandem-repeat region;
until reaching converged genotype probabilities of tandem-repeat genotypes, iteratively:
determining, for the genomic sample and utilizing a stutter model, expected genotype probabilities of candidate tandem-repeat genotypes based on differing numbers of nucleotide repeat units in the spanning nucleotide reads; and
updating parameters of the stutter model based on the expected genotype probabilities; and
determining a genotype call from the candidate tandem-repeat genotypes that the genomic sample comprises one or more tandem-repeat alleles at the tandem-repeat region based on the converged genotype probabilities.

39. The method of claim 38, wherein determining the expected genotype probabilities of tandem-repeat genotypes comprises performing an expectation stage of an expectation-maximization (EM) algorithm by:
generating, utilizing the stutter model, read probabilities of nucleotide reads originating from a set of candidate tandem-repeat alleles; and
determining, utilizing the stutter model, the expected genotype probabilities based on the read probabilities.

40. The method of claim 39, wherein updating the parameters of the stutter model further comprises performing a maximization stage of an EM algorithm by:
generating, utilizing the stutter model, updated allele probabilities of candidate tandem-repeat alleles among the spanning nucleotide reads based on the expected genotype probabilities; and
modifying the parameters of the stutter model to maximize a total probability of the expected genotype probabilities based on the updated allele probabilities.
